(19) United States
(12) Patent Application Publication
(10) Pub. No.: US 2014/0309275 A1
(43) Pub. Date: Oct. 16, 2014

(54) Sphingosine Analogs, Compositions, and Methods Related Thereto

(71) Applicant: Emory University, Atlanta, GA (US)

(72) Inventors: Dennis C. Liotta, Atlanta, GA (US); Jason Holt, JR., Atlanta, GA (US); Michael G. Natchus, Alpharetta, GA (US); Mark T. Baillie, Lausanne (CH); Eric J. Miller, Atlanta, GA (US); Mary R. Galinski, Stone Mountain, GA (US)

(21) Appl. No.: 14/348,452

(22) PCT Filed: Sep. 27, 2012
(86) PCT No.: PCT/US2012/057448

§ 371 Date: Jun. 3, 2014

Related U.S. Application Data
(60) Provisional application No. 61/540,559, filed on Sep. 29, 2011.

(51) Int. Cl. C07D 207/08; A61K 45/06; A61K 31/40; C07C 215/10; A61K 31/133

(52) U.S. Cl. CPC C07D 207/08; C07C 215/10; A61K 31/133; A61K 31/40; A61K 45/06
USPC 514/428; 554/109; 514/669; 548/570

(57) ABSTRACT

The disclosure relates to compounds, pharmaceutical compositions, and methods of treating or preventing disease. In certain embodiments, the disclosure relates to methods of treating an infection or cancer comprising administering a pharmaceutical composition disclosed herein to a subject in need thereof. In a typical embodiment, one administers a pharmaceutical composition comprising sphingosine or a sphingosine analog to a subject at risk for, exhibiting symptoms of or diagnosed with a malaria infection.

### CROSS REFERENCE TO RELATED APPLICATIONS

This application claims priority to U.S. Provisional Application No. 61/540,559 filed Sep. 29, 2011, hereby incorporated by reference in its entirety.

### BACKGROUND

Many microorganisms, microbial toxins, and viruses bind to cells via sphingolipids. Specific organisms that have been reported as binding to sphingolipids include cholera toxin (ganglioside GM1), Shiga-like toxin 2e (globotriaosylceramide, Gb3), and Clostridium botulinum type B neurotoxin (to synaptotagmin II associated with gangliosides GT1b/GD1a). Furthermore, many bacteria utilize sphingolipids to adhere to cell. Examples known in the art include Escherichia coli (galactosylceramide), Haemophilus influenza (gangliotetraosylceramide and gangliotriosylceramide), Helicobacter pylori (gangliotetraosylceramide, gangliotriaosylceramide, sulfatides and GM3), Borrelia burgdorferi (galactocerebroside; Virulent strain 297; glycosylceramide, lactosylceramide, and galactosylgloboside), and Pseudomonas aeuroginosa and Candida albicans (asialo GM1).

Sphingolipids not only help define the structural properties of membranes, but also play roles in cell-cell and cell-substratum interactions, and help regulate growth and differentiation by a variety of mechanisms, such as inhibition of growth factor receptor kinases and effects on numerous cellular signal transduction systems. The current paradigm for the action of sphingolipids in cell regulation is that complex sphingolipids are important in membrane structure, especially specialized membrane functions such as are found in calveolae. The lipid backbones (ceramide, sphingosine and sphingosine 1-phosphate) function as “second messengers” to affect protein kinases, phosphoprotein phosphatases, ion transporters, and other regulatory machinery. As examples, tumor necrosis factor-alpha, interleukin 1 beta, and nerve growth factor induce sphingomyelin hydrolysis to ceramide as a second messenger; other agonists, such as platelet-derived growth factor, trigger further hydrolysis of ceramide to sphingosine, and activate sphingosine kinase to form sphingosine 1-phosphate. Depending on the cell type, these metabolites can either stimulate or inhibit growth. While the details of growth regulation by ceramide, sphingosine, and sphingosine 1-phosphate are still being uncovered, depending on the system, it appears to involve calcium mobilization from intracellular stores, and activation of the MAP (and Jun) kinase pathways and transcription factors, and in some cases, induction of apoptosis.

Improved anti-malarials are needed in the face of widespread drug resistance against P. falciparum and to provide alternative compounds for inclusion in future combination therapies. The sphingolipid pool is an important factor in modulating vital eurokaryotic cellular functions, including those of the protozoan genus, Plasmodium. In malaria infections, membrane formation is important for the growth and development of the Plasmodium parasite inside host erythrocytes. New membraneous structures are formed beginning as the parasite invades these host cells with the formation of a parasitophorous vacuole and then throughout the red blood cell (RBC) cytoplasm to create trafficking networks, important for the import and export of nutrients and waste products as the parasite grows to a trophozoite and then multiplies through the schizont stages of development. The Plasmodium sphingolipid metabolic pathway is known to involve several enzymes (e.g. sphingomyelin synthase, glucosylceramide synthase and two sphingomyelinases) and compounds. Fumonisin B or phenyl-2-palmitoylamino-3-morpholino-1-propanol (PPMP) have been shown to interfere with the sphingolipid metabolism of P. falciparum. See Tilley et al., Traffic, 2008, 9 (2), 187-97; Lauer et al., Mol Biochem Parasitol, 2001, 115 (2), 275-81; Lauer et al., Proc Natl Acad Sci, 1995, 92 (20), 9181-5; Couto et la., Eur J Biochem, 2004, 271 (11), 2204-14; Hanada et al., Biochem J, 2000, 346 Pt 3, 671-7; and Gerold & Schwarz, Mol Biochem Parasitol, 2001, 112 (1), 29-37

The fungal-derived natural product mycotoxin, fumonisin B1 (FB1, FIG. 1), is a 1-deoxy, 5-hydroxy sphingolipid that has activity as an inhibitor of ceramide synthase in vitro. However, FB1 does not block the intraerythrocytic development of the malaria parasite and, displays weak anti-parasitic activity, indicating that inhibition of de novo synthesis of ceramide by itself is not a viable therapeutic strategy for this parasite. In contrast, the synthetic sphingolipid analog, threo-PPMP which has been shown to inhibit sphingomyelin synthase activity in RBCs with an IC₅₀ of 0.85 μM, is able to arrest growth of the parasite. However, threo-PPMP appears to exhibit only cytostatic effects, and no animal studies have been reported. Several synthetic analogs of threo-PPMP have been reported that display an increase in anti-parasitic activity. See Labaied et al., Malar J, 2004, 3, 49. Scyphostatin has been identified as a low micromolar inhibitor of neutral sphingomyelinase, which also inhibits intraerythrocytic parasite replication in vitro. See Hanada et al., J Exp Med, 2002, 195 (1), 23-34. This enzyme may release phosphocholine and/or phospholipids from the host cell lipids, thereby providing the substrates for the synthesis of sphingomyelin by the parasite's sphingomyelin synthase associated with membranous structures in the erythrocyte cytoplasm. However, the lack of stability associated with scyphostatin in the solid state greatly limits its potential as a drug candidate. Thus, there is a need to identify improved compounds that effectively block sphingomyelin synthase activity and/or glycosphingolipid biosynthesis for use as anti-malarial agents.

U.S. Pat. No. 6,610,835 discloses sphingosine analogues. It also discloses methods of treating infections and cancer.

### SUMMARY

In certain embodiments, the disclosure relates to sphingosine analogs and compounds disclosed herein in pharmaceutical compositions for the treatment of infections and cancer. In certain embodiments, the infectious disease is caused by a protozoa, viral, bacterial, or fungal infection. In certain embodiments, the disease is malaria, amoebiasis, giardiasis, toxoplasmosis, cryptosporidiosis, trichomoniasis, leishmaniasis, sleeping sickness, or dysentery.

In certain embodiment, the disclosure relates to pharmaceutical compositions comprising a compound disclosed herein or salt thereof and a pharmaceutically acceptable excipient. In certain embodiments, the pharmaceutical composition further comprises a second therapeutic agent such as an anti-malaria agent, anti-viral agent, antibiotic, or anti-cancer agent.

In certain embodiments, the disclosure relates to methods of treating or preventing an infection comprising administering an effective amount of a compound disclosed herein to a subject in need thereof. Typically, the subject is diagnosed with or at risk of a malaria infection. The subject may also be diagnosed with or at risk of an infection from a virus, bacteria, fungi, protozoa, or parasite. The subject may be administered the compound in combination with a second therapeutic agent such as anti-malaria agent, anti-viral agent, or antibiotic.

In certain embodiments, the disclosure relates to methods of treating or preventing cancer comprising administering an effective amount of a compound disclosed herein to a subject in need thereof. The cancer may be selected from bladder cancer, lung cancer, breast cancer, melanoma, colon and rectal cancer, non-hodgkin lymphoma, endometrial cancer, pancreatic cancer, kidney cancer, prostate cancer, leukemia, thyroid cancer, and brain cancer. The compound may be administered in combination with a second anticancer agent.

In certain embodiments, the disclosure relates to a pharmaceutical composition comprising a compound as described herein or a pharmaceutically acceptable salt or prodrug thereof. In certain embodiments, the composition further comprises a second active pharmaceutical ingredient.

In certain embodiments, the disclosure relates to the use of a compound as described herein in the manufacture of a medicament for the treatment of an infectious disease or cancer.

In certain embodiments, the disclosure contemplates the use of fluorinated compounds for imaging. In certain embodiment, a method is contemplated comprising administering compounds disclosed herein to a subject or sample, exposing an area of the subject or the sample to a magnetic field and a pulse of radio frequencies. Method typically comprises detecting nuclear magnetic resonance frequencies, such as from fluorine, hydrogen, and/or carbon. It is also contemplated that the method includes creating an image from the detected resonance frequencies.

### BRIEF DESCRIPTION OF THE FIGURES

FIG. 1 illustrates sphingolipid modulators and anti-malarial agents.

FIG. 2 shows data on the effect of ESPD-0507 on the development of P. falciparum W2 strain at 28 and 48 hours post incubation vs. untreated parasites (RPMI).

FIG. 3 shows in vivo data for select compounds. A. Both Enigmol and N-Methylenigmol show dose proportional PK and long plasma half-lives in mice. B. Enigmol shows plasma accumulation after 5 daily doses and 3-4 fold higher levels in red blood cells. NME=N-Methylenigmol.

FIG. 4 illustrates the preparation of embodiments of the disclosure.

FIG. 5 illustrates the preparation of embodiments of the disclosure.

FIG. 6 illustrates the preparation of embodiments of the disclosure.

FIG. 7 illustrates the preparation of embodiments of the disclosure.

FIG. 8 illustrates the preparation of embodiments of the disclosure.

FIG. 9 illustrates the preparation of embodiments of the disclosure.

FIG. 10 illustrates the preparation of embodiments of the disclosure.

FIG. 11 shows data for average tumor volumes (top) and body weights (bottom) for Enigmol, ESPD-00561, and ESPD-01406 in a mouse xenograft cancer model.

FIG. 12 shows data for average tumor volumes (top) and body weights (bottom) for Enigmol and ESPD-01183 in a mouse xenograft cancer model.

FIG. 13 shows data for rat tissue distribution of Enigmol, ESPD-01183, and ESPD-01406.

### DETAILED DESCRIPTION

### Terms

When describing the compounds for use in the disclosure, the terms used are to be construed in accordance with the following definitions, unless a context dictates otherwise.

As used herein, “alkyl” means a noncyclic straight chain or branched, unsaturated or saturated hydrocarbon such as those containing from 1 to 10 carbon atoms, while the term “lower alkyl” has the same meaning as alkyl but contains from 1 to 6 carbon atoms. The term “higher alkyl” has the same meaning as alkyl but contains from 7 to 20 carbon atoms. In certain embodiments, the disclosure contemplates that alkyl refers to lower alkyl or higher alkyl. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

As used herein, “heteroaryl” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra and —S(═O)2ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g. patient) is cured and the disease is eradicated. Rather, embodiments, of the present disclosure also contemplate treatment that merely reduces symptoms, and/or delays disease progression.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In preferred embodiment the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Subject” refers any animal, preferably a human patient, livestock, or domestic pet.

The term “malaria” as used herein refers to an infectious disease, also known as ague or marsh fever, typically caused by a protistan parasite of the genus Plasmodium, suitably, P. falciparum, P. vivax, P. ovale or P. malariae. This parasite is transmitted primarily by female Anopheles mosquitoes. Plasmodium invades and consumes the red blood cells of its hosts, which leads to symptoms including fever, anemia, and in severe cases, a coma potentially leading to death.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis.

### Sphingosine Analogs

In certain embodiments, the sphingosine analogs may be compounds comprising formula I:

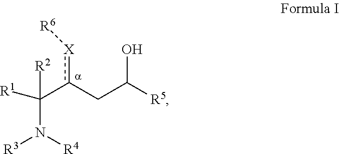

prodrugs, esters, or salts thereof wherein,

X is O or N;

the dotted lines are an optional bond to provide an absent, single, or double bond provided that if X is O and the bond between the X and the alpha carbon is a double bond, then R⁶ is absent;

R¹ and R² are independently hydrogen or alkyl optionally substituted with one or more, the same or different, R⁷, or R¹ and R² form a 3-7 membered carbocyclic or heterocyclic ring optionally substituted with one or more, the same or different, R⁷;

R³ and R⁴ are independently hydrogen, alkyl, or alkanoyl optionally substituted with one or more R⁷, or R¹ and R³ and the atoms which they are attached form a 4-7 membered heterocyclic ring optionally substituted with one or more, the same or different, R⁷;

R⁵ is a higher alkyl or other lipophilic moiety optionally substituted with one or more, the same or different, R⁷;

R⁶ is hydrogen or alkyl wherein R⁶ is optionally substituted with one or more, the same or different, R⁷;

R⁷ alkyl, halogen, nitro, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁷ is optionally substituted with one or more, the same or different, R⁸; and

R⁸ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, compounds of Formula I have Formula IA:

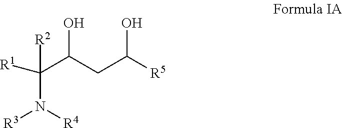

prodrugs, esters, or salts thereof wherein,

R¹ and R² are independently hydrogen or alkyl optionally substituted with one or more, the same or different, R⁷;

R³ and R⁴ are independently hydrogen, alkyl, or alkanoyl optionally substituted with one or more R⁷, or R¹ and R³ and the atoms which they are attached form a 4-7 membered heterocyclic ring optionally substituted with one or more, the same or different, R⁷;

R⁵ is a higher alkyl or other lipophilic moiety optionally substituted with one or more, the same or different, R⁷;

R⁶ is hydrogen or alkyl wherein R⁶ is optionally substituted with one or more, the same or different, R⁷;

R⁷ alkyl, halogen, nitro, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁷ is optionally substituted with one or more, the same or different, R⁸; and

R⁸ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, R¹ and R² are alkyl optionally substituted with one or more, the same or different, R⁷.

In certain embodiments, R¹ and R² form a 3-6 membered carbocyclic ring optionally substituted with one or more, the same or different, R⁷.

In certain embodiments, R⁵ is an unsaturated higher alkyl.

In certain embodiments, R⁵ is a higher alkyl substituted with one or more halogen optionally substituted with one or more, the same or different, R⁷.

In certain embodiments, R⁵ is alkyl substituted with one or more fluorine optionally substituted with one or more, the same or different, R⁷.

In certain embodiments, R³ is hydrogen and R⁴ is alkyl optionally substituted with one or more, the same or different, R⁷.

In certain embodiments, the sphingosine analog is selected from
2-aminooctadecane-3,5-diol;
(2S,3S,5S)-2-aminooctadecane-3,5-diol;
(2S,3R,5S)-2-aminooctadecane-3,5-diol;
2-(methylamino)octadecane-3,5-diol;
(2S,3R,5S)-2-(methylamino)octadecane-3,5-diol;
2-(dimethylamino)octadecane-3,5-diol;
(2R,3S,5S)-2-(dimethylamino)octadecane-3,5-diol;
1-(pyrrolidin-2-yl)hexadecane-1,3-diol;
(1S,3S)-1-((S)-pyrrolidin-2-yl)hexadecane-1,3-diol;
2-amino-11,11-difluorooctadecane-3,5-diol;
(2S,3 S,5 S)-2-amino-11,11-difluorooctadecane-3,5-diol;
11,11-difluoro-2-(methylamino)octadecane-3,5-diol;
(2S,3 S,5 S)-11,11-difluoro-2-(methylamino)octadecane-3,5-diol;
N-((2S,3S,5S)-3,5-dihydroxyoctadecan-2-yl)acetamide;
N-((2S,3S,5S)-3,5-dihydroxyoctadecan-2-yl)palmitamide; and
esters, prodrugs, and salts thereof.

In certain embodiments, the sphingosine analog is a compound selected from:
1-(1-aminocyclopropyl)hexadecane-1,3-diol;
(1S,3R)-1-(1-aminocyclopropyl)hexadecane-1,3-diol;
(1S,3S)-1-(1-aminocyclopropyl)hexadecane-1,3-diol;
2-amino-2-methyloctadecane-3,5-diol;
(3S,5S)-2-amino-2-methyloctadecane-3,5-diol;
(3S,5R)-2-amino-2-methyloctadecane-3,5-diol;
(3S,5S)-2-methyl-2-(methylamino)octadecane-3,5-diol;
2-amino-5-hydroxy-2-methyloctadecan-3-one;
(Z)-2-amino-5-hydroxy-2-methyloctadecan-3-one oxime; and
prodrugs, esters, or salts thereof.

In certain embodiments, the sphingosine analog is a compound selected from:
(2S,3R,5R)-2-amino-6,6-difluorooctadecane-3,5-diol;
(2S,3S,5R)-2-amino-6,6-difluorooctadecane-3,5-diol;
(2S,3S,5S)-2-amino-6,6-difluorooctadecane-3,5-diol;
(2S,3R,5S)-2-amino-6,6-difluorooctadecane-3,5-diol;
(2S,3S,5S)-2-amino-18,18,18-trifluorooctadecane-3,5-diol; and
prodrugs, esters, or salts thereof.

In some embodiments the disclosure relates to compositions comprising a compound of formula I in diastereomeric excess or substantially pure form, wherein the diastereomeric excess is greater than 60%, 70%, 80%, 90%, 95% or 99%.

In certain embodiments the compound of Formula I is selected from Formula IB, or IC.

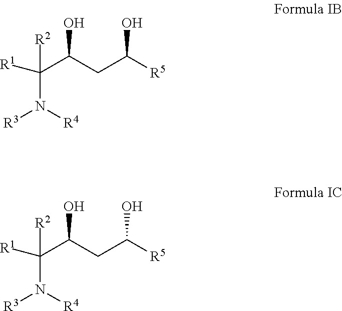

prodrugs, esters, or salts thereof wherein, R¹-R⁵ are described herein.

In a typical embodiment, R¹ is methyl optionally substituted with one or more, the same or different, R⁷.

In certain embodiments, R² is hydrogen or alkyl optionally substituted with one or more, the same or different, R⁷.

In certain embodiments, R³ is hydrogen, alkyl, or alkanoyl optionally substituted with one or more, the same or different, R⁷.

In certain embodiments, R¹ and R³ and the atoms which they are attached to form a 5 membered ring optionally substituted with one or more, the same or different, R⁷.

In certain embodiments, R⁴ is hydrogen optionally substituted with one or more, the same or different, R⁷.

In certain embodiments, R⁵ is —(CH₂)₁₂CH₃ optionally substituted with one or more, the same or different, R⁷.

In certain embodiments, compounds of Formula I have Formula ID:

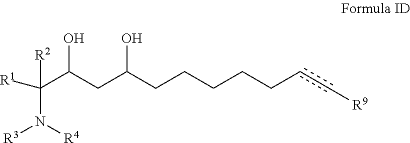

prodrugs, esters, or salts thereof wherein,

the dotted lines are an optional bond to provide an single, double, or triple bond:

R¹ and R² are independently hydrogen or alkyl optionally substituted with one or more, the same or different, R⁷, or R¹ and R² form a 3-7 membered carbocyclic or heterocyclic ring optionally substituted with one or more, the same or different, R⁷;

R³ and R⁴ are independently hydrogen, alkyl, or alkanoyl optionally substituted with one or more R⁷, or R¹ and R³ and the atoms which they are attached form a 4-7 membered heterocyclic ring optionally substituted with one or more, the same or different, R⁷;

R⁷ alkyl, halogen, nitro, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁷ is optionally substituted with one or more, the same or different, R⁸;

R⁸ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl;

R⁹ is a alkyl optionally substituted with one or more, the same or different, R¹⁰;

R¹⁰ alkyl, halogen, nitro, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹⁰ is optionally substituted with one or more, the same or different, R¹¹; and

R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, R⁹ is alkyl optionally substituted with one or more, the same or different halogen, such as fluorine.

In certain embodiments, the optional dotted lines provide a double bond in the cis configuration.

In certain embodiments, the optional dotted lines provided a double bond in the trans configuration.

### Compound Activity Evaluation

It has been discovered that certain sphingoid base analogs inhibit the normal growth and development of both P. falciparum and P. knowlesi infected erythrocytes, and morphological studies suggest that the inhibitory affect begins early in the ring stage of the parasite's development. Certain compounds disclosed herein can inhibit the growth and replication of the Plasmodium infected red blood cells (RBCs) and offer the potential for use as acute, anti-malaria therapies.

The [³H]-labeled hypoxanthine uptake method was utilized to assay the effectiveness of our sphingoid base analogs in vitro for their ability to inhibit the growth and replication of malaria parasites in RBCs. Preliminary dose-response assays were conducted using two strains of P. falciparum, the chloroquine resistant (CQ-R) W2 strain, and the chloroquine susceptible (CQ-S) D6 strain. The simian malaria parasite P. knowlesi has also been recognized as a human pathogen of public health concern in South East Asia, and it can be cultured effectively in vitro. This species was therefore included to probe the potential of multi-species efficacy within this compound class. The data were generated with the known antimalarial drugs chloroquine and mefloquine as controls.

Table 1 shows comparative IC₅₀ concentrations for P. falciparum W2 and D6 strains and P. knowlesi when tested for their sensitivity to Enigmol and ten selected analogs. Enigmol and other analogs tested exhibited anti-malarial activity on both W2 and D6 strains at less than 10 μM. There was a distinct and observable structure activity relationship (SAR) between Enigmol and its analogs that ranged over roughly one order of magnitude, and was quantitatively similar for both P. falciparum strains. Enigmol was found to have an IC₅₀ between 7 and 12 μM. There was modest potency difference among the C-3 and C-5 diastereomers.

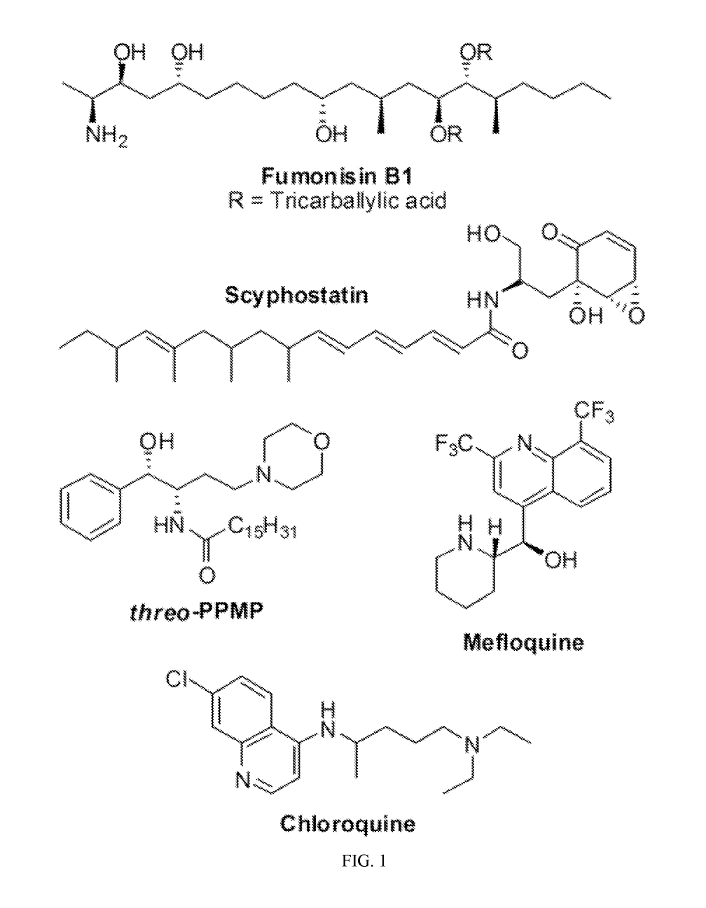

TABLE 1
Compound SAR data.

| | | IC₅₀ (μM) | | |
|---|---|---|---|---|
| Compound | Structure | W2 | D6 | P. knowlesi |
| Enigmol ESPD-0500 | 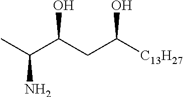 | 7.37 ± 1.5 | 12.64 ± 2.4 | 10.48 ± 0.38 |
| ESPD-0505 | 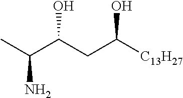 | 9.22 ± 1.6 | 17.8 ± 1.1 | 20.32 ± 2.0 |
| ESPD-0559 | 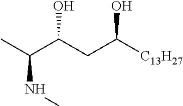 | 8.09 ± 0.24 | 14.56 ± 0.64 | NT |
| ESPD-0503 | 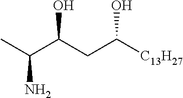 | 8.17 ± 1.8 | 12.7 ± 1.2 | 17.61 ± 1.8 |
| N-Methyl- enigmol ESPD-0507 | 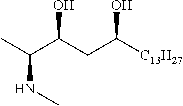 | 1.58 ± 0.13 | 4.7 ± 0.1 | 1.72 ± 0.01 |
| ESPD-0513 | 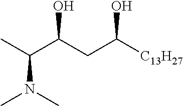 | 4.42 ± 1.1 | 7.0 ± 0.6 | 6.83 ± 0.09 |
| ESPD-0522 | 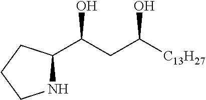 | 4.39 ± 1.5 | 9.7 ± 1 | 6.25 ± 0.27 |
| ESPD-0563 | 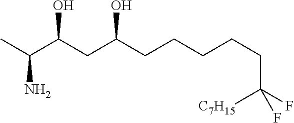 | 5.58 ± 0.01 | 12.67 ± 0.32 | NT |
| ESPD-0564 | 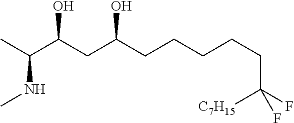 | 2.37 ± 0.25 | 7.34 ± 0.14 | NT |
| ESPD-0506 | 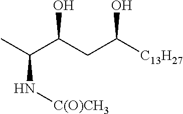 | 13.11 ± 3.7 | 11.1 ± 0.5 | NE |
| ESPD-0514 | 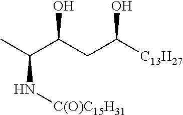 | NE | NE | NE |
| Chloroquine | See FIG. 1 | 0.18 | 0.02 | 0.01 |
| Mefloquine | See FIG. 1 | 0.02 | 0.08 | 0.04 |

N-methylation appears to increase potency. N-Methylenigmol (ESPD-0507) was particularly potent against all three malaria strains and this is within an order of magnitude in potency relative to the known positive control, chloroquine, in the CQ-R W2 strain. N-Methyl analogs ESPD-0559 and -0564 also displayed increase potency; pyrrolidine analog ESPD-0522 constrains C1 and the C2 amine functionality into a ring and demonstrates that this modification is well tolerated. Fluorination of the side chain, such as with analog ESPD-0563 and its N-methyl equivalent, ESPD-0564, is also well tolerated. N-acylation of Enigmol (ESPD-0506) or the N-palmitoyl analog (ESPD-0514), displayed no activity against W2 and D6 P. falciparum strains or P. knowlesi. The analogs were typically more effective against the CQ-R W2 strain, suggesting that the sphingolipid analogs may be independent from the mechanisms that impart chloroquine resistance. These data also suggest that sphingolipid analogs function by different inhibitory mechanism providing a malaria therapy administering multiple agents.

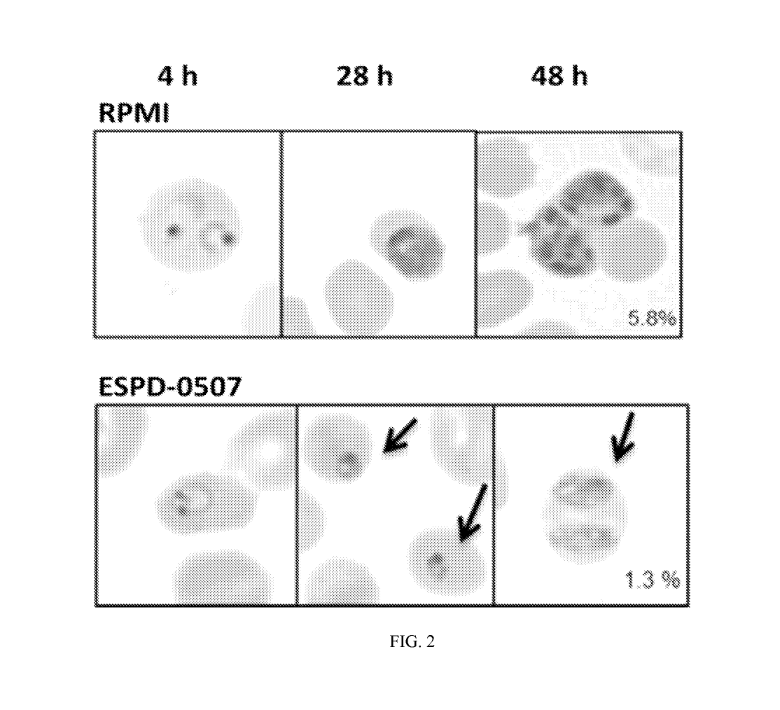

Upon preliminary examination, morphological changes were detected in the P. falciparum W2 strain parasites after incubation of the cultures with N-Methylenigmol (ESPD-507). The effect was evident between 28 h and 48 h of incubation, with a quantitatively high percentage of various aberrant or stunted forms of ring-stage and trophozoite-stage parasites found on Giemsa-stained thin blood smears (see black arrows in FIG. 2 at 28 h highlighting the presence of abundant ring-stage parasites and at 48 h, noting a vacuolated appearance of trophozoite-stage parasites). These findings can be contrasted with the normal growth, development, and maturation of the parasites without the drug. This inhibitory effect occurs at the ring state of development, as is also evidenced by the fact that the test compound resulted in an accumulation of the younger stage parasites (i.e., without their natural growth and development), while the control culture developed normally through the different growth stages.

By the 48 hour time point, the parasitemia increased from 1% to 5.8+/−0.5% with healthy appearing parasites in the control samples, as expected, while it remained at 1.3+/−0.3% and with aberrant parasite forms in the sample incubated with N-Methylenigmol (Table 2). The relative percentages of rings, trophozoites and schizonts in the cultures with and without the ESPD 507 compound were 41.9%, 39.2% and 18.9% versus 87.9%, 4.2% and 8.05, respectively. These data reflect the fact that the parasites that were incubated with N-Methylenigmol did not progress with normal development (from ring stage to trophozoites, to multinucleated schizonts and newly invaded RBC's), while the untreated parasites progressed through schizogony with a high percentage of newly invaded RBCs and the normal development new young ring-stage parasites.

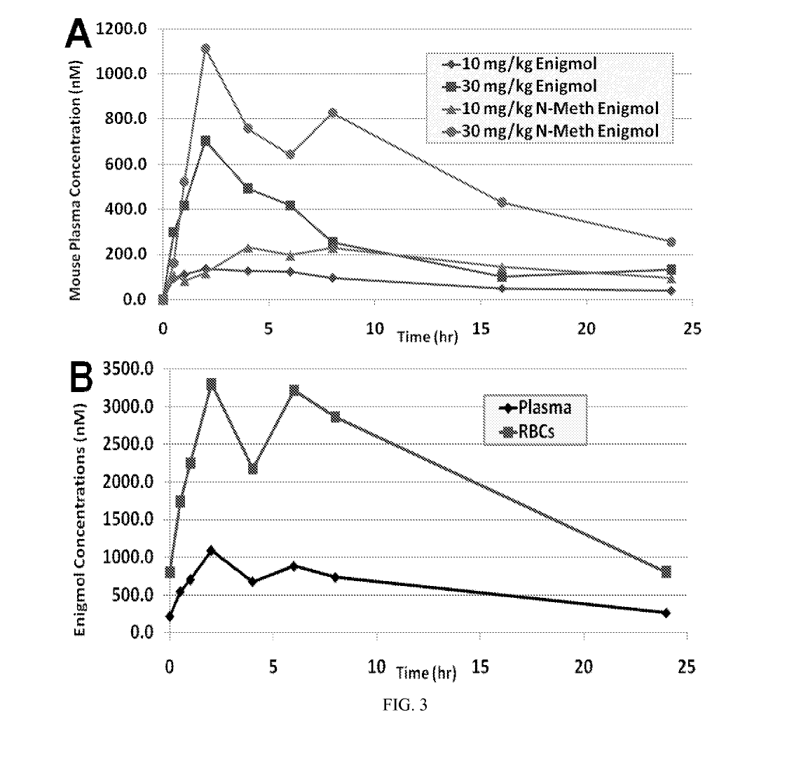

Enigmol and its N-methyl-analog possess attractive drug-like properties that provide sustained plasma and tissue levels that are in the micromolar range. Both compounds displayed large volumes of distribution, suggesting substantial tissue accumulation. Mice were dosed with Enigmol once per day orally at 30 mg/kg and drug levels were followed in both plasma and RBCs after the 5^{th} day of dosing (FIG. 3B). Plasma levels increased by about 30% from accumulation, and that drug concentrations in the RBCs were roughly 3-fold higher than plasma levels at any given time, supporting the idea that Enigmol partitions into membranous tissues.

Conjugating the compounds to Bovine Serum Albumin (BSA) was done to increase their solubility in the assays. The [³H]-labeled hypoxanthine uptake assay was followed as described in the methods section. The results for Enigmol and N-Methylenigmol, shown in Table 2, indicate that formation of BSA complexes with sphingolipid analogs provides for slightly greater efficacy in the assay, presumably due to the resolution of solubility or aggregation. Analogs were modified at C2. Dimethyl and cyclopropyl analogs at C2 eliminate the chirality of the center. ESPD-1158 was determined to have an IC₅₀ of 250 nM

TABLE 2
Compound SAR data.

| | | IC₅₀ (μM) | |
|---|---|---|---|
| Compound | Structure | W2 | D6 |
| Enigmol ESPD-0500 | 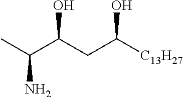 | 9.2 ± 2 | 5.3 ± 1 |
| N-Methyl- enigmol ESPD-0507 | 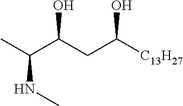 | 0.9 ± 0.35 | 1.7 ± 1 |
| ESPD-0859 | 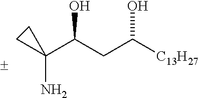 | 7.2 ± 4 | 11.2^{a} |
| ESPD-0860 | 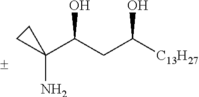 | 9.9 ± 7 | 5.5^{a} |
| ESPD-0560 | 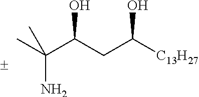 | 4.67 ± 1 | 5.2 ± 3 |
| ESPD-0561 | 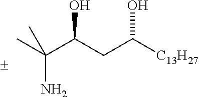 | 7.43^{a} | 4.1 ± 0.7 |
| ESPD-1158 | 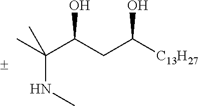 | 0.25 | 0.7 ± 0.5 |
| ESPD-0562 | 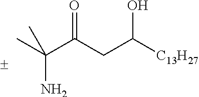 | 2.6 ± 1.5 | 2.5 ± 0.1 |
| ESPD-0858 | 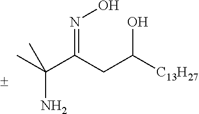 | 10 ± 2.7 | 7.44 |
| Chloroquine | See FIG. 1 | 0.093 | 0.011 |
| Mefloquine | See FIG. 1 | 0.004 | 0.03 |

Prostate cancer cells were treated with enigmol or analogue for 24 h, and cytotoxicity was assessed by WST-1 assay. cLogPs were calculated with QuikProp on Maestro. Potency in PC-3 and LNcAP cell lines were evaluated, and IC₅₀ values are provided in the table below.

| | | | LNCaP IC₅₀ | PC-3 IC₅₀ |
|---|---|---|---|---|
| Compound | Structure | cLogP | (μM) | (μM) |
| Enigmol | 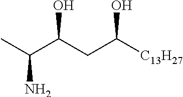 | 3.47 | 13 | 10 |
| ESPD-01406 | 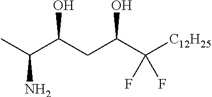 | 3.94 | 10 | 9 |
| 01407 | 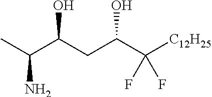 | 3.94 | 10 | 9 |
| 01408 | 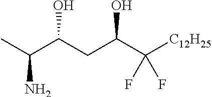 | 3.94 | 10 | 9 |
| 01409 | 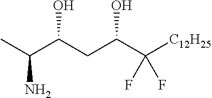 | 3.94 | 10 | 10 |
| ESPD-00563 | 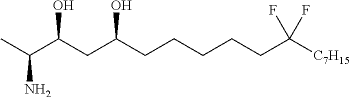 | 3.82 | 18 | 13 |
| ESPD-01183 | 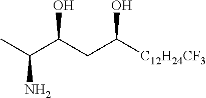 | 4.13 | 13 | 13 |
| ESPD-01191 | 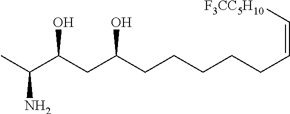 | 4.17 | 22 | 43 |
| ESPD-01192 | 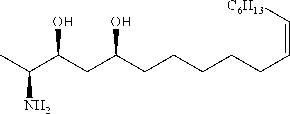 | 3.49 | 19 | 25 |
| ESPD-01186 | 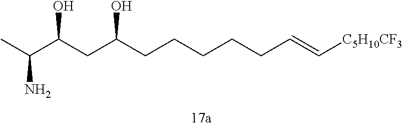 | 4.10 | 14 | 27 |
| ESPD-01187 | 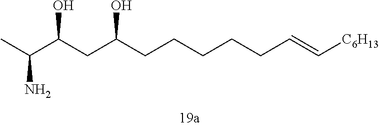 | 3.38 | 19 | 33 |
| ESPD-01184 | 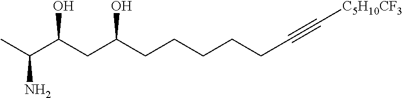 | 3.70 | >100 | 92 |
| ESPD-01185 | 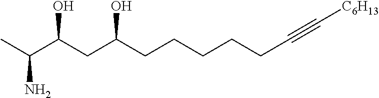 | 3.42 | >100 | >100 |
| ESPD-00858 | 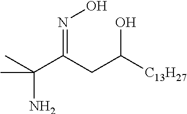 | 3.48 | 24 | 24 |
| ESPD-00560 | 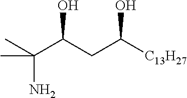 | 3.87 | 23 | 10 |
| ESPD-00561 | 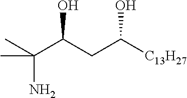 | 3.87 | 25 | 10 |

### Methods of Preparing Compounds

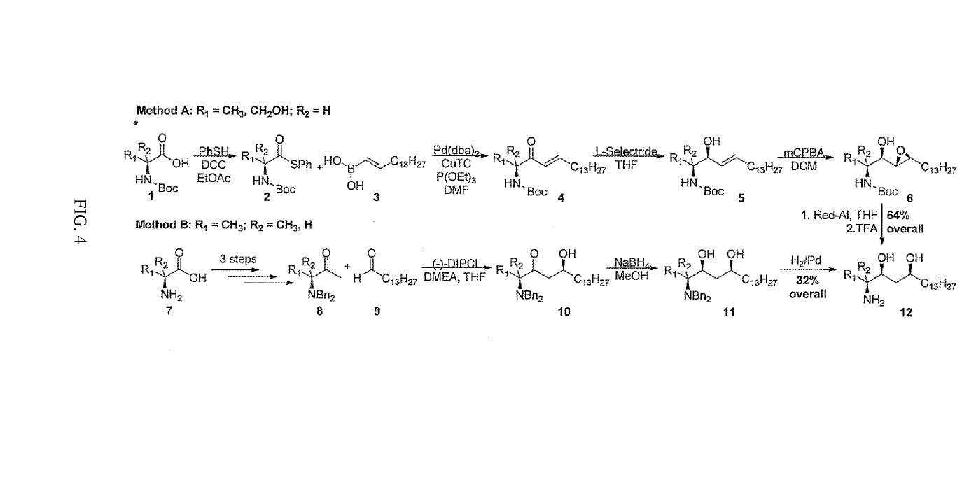

Synthetic routes for preparing sphingolipids analogs are provided in FIGS. 4 to 9. 1-Deoxy-5-hydroxy-sphingoid base analogs retain many of the physical characteristics of the natural sphingolipids but lack a C-1 primary hydroxyl group. Although it is not intended that certain embodiments be limited by any particular mechanism, it is believed that by trans-locating the C-1 hydroxyl group to the C-5 position, one would maintain similar compound hydrophobicity and enzymatic recognition while eliminating the possibility for phosphorylation of the C-1 hydroxyl group by sphingosine kinase (SK). This is desirable since the phosphate intermediates formed by SK are subject to catabolic degradation and also possess undesirable pro-mitogenic and anti-apoptotic properties.

The syntheses of Enigmol, its C-3, C-5 diastereomers, and N-Methylenigmol are provided in FIG. 4. Pyrrolidine analog ESPD-0522 was prepared using similar reaction conditions (FIG. 4, Method B) starting from L-proline. N-Acylenigmol (ESPD-0506) and N-Palmitoylenigmol (ESPD-0514) were prepared from Enigmol using standard N-acylation conditions. Difluoro analogs ESPD-0563 and -00564 were prepared using the route described in Scheme 1, Method B. N,N-dimethylenigmol (ESPD-0513) was prepared by treatment of Enigmol with aqueous 37% formaldehyde, and then with sodium cyanoborohydride in acetate buffer.

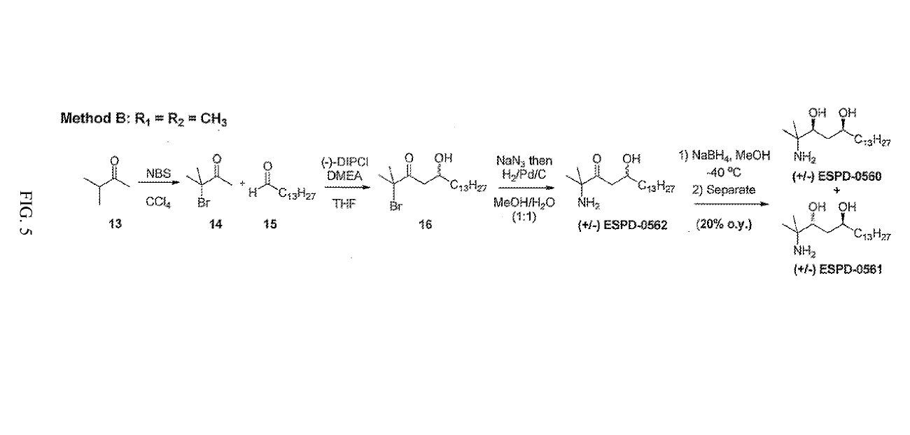

The oxime analog ESPD-0858 was available via reaction of ESPD-0562 with HONH₂ HCl. The related cyclopropyl analogs were prepared similarly using Method B and starting with cyclopropyl methylketone. See FIG. 5. Unfortunately elimination of chirality at C2 had a detrimental effect on the stereoselectivity of the aldol reaction in both the gem-dimethyl and cyclopropyl cases.

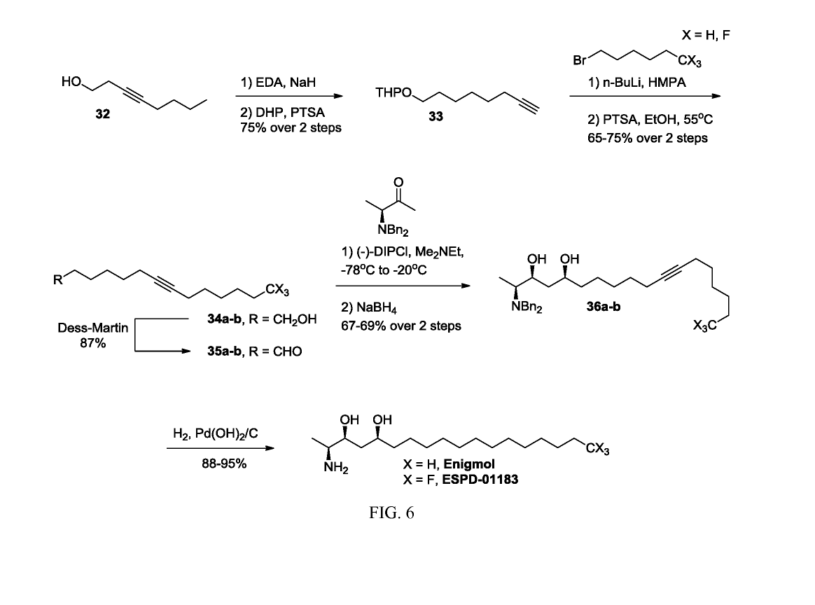

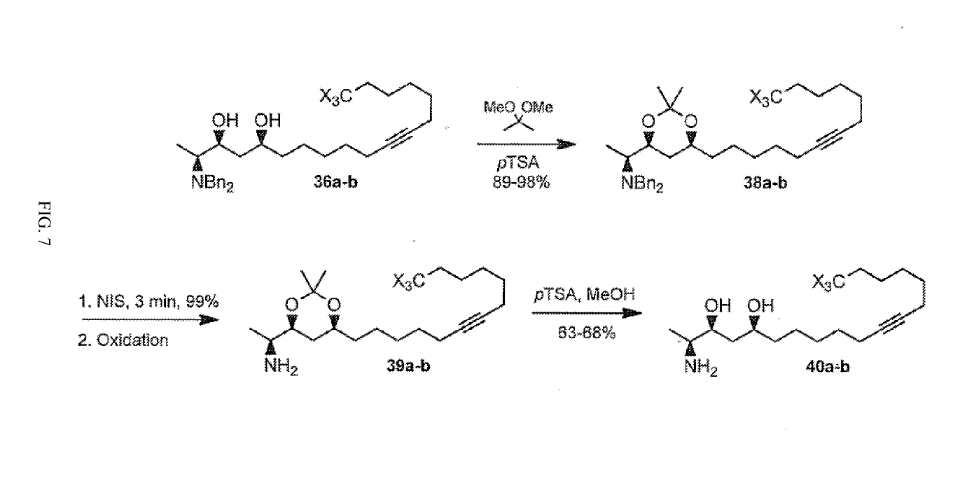

The synthesis of the tail modified enigmols utilized a boron-mediated aldol. See FIG. 6. The synthesis utilized 3-octyne-1-ol (32), which was subject to the alkyne zipper reaction with ethylenediamene (EDA) and sodium hydride to furnish the terminal alkyne, and subsequent protection of the alcohol with a THP group to give 36. The protected alkyne then underwent a base mediated coupling reaction with each of the alkyl-bromides (1-bromohexane or 1-bromo-6-trifluoromethylhexane), followed by immediate deprotection of the THP group before purification to greatly simplify chromatographic separation. The terminal alcohol of 34a-b were then oxidized with Dess-Martin periodinane to consistently give the aldehyde. The aldehydes 35a-b were used (within 24 hours typically) in the aldol step, followed by work up and reduction to give the diastereomer for each of the compounds 36a-b. To obtain the first, saturated analog, the N,N-dibenzyl, alkynyl CF3 intermediate 36b was subjected to standard hydrogenolysis conditions to give the C18 trifluoromethyl enigmol 37. IBX and Dess-Martin periodinane oxidative removal was found to be desirable if allowed to react for 90 to 110 minutes and not more as byproducts that were not present in small scale began to appear. See FIG. 7. The alkynyl targets 40a and 40b were realized by deprotection of the acetonide under acidic conditions.

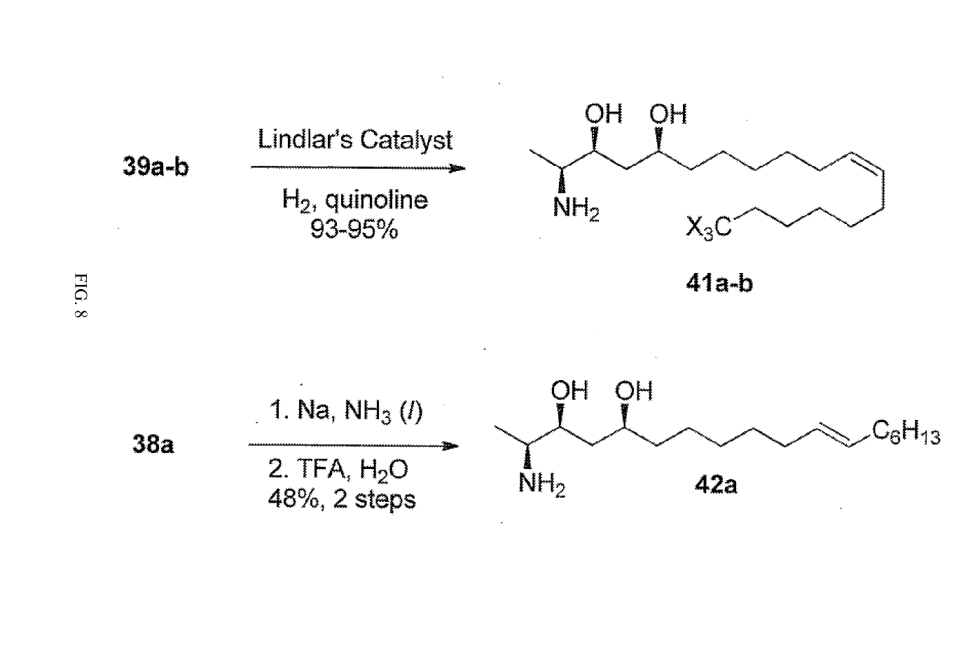

To attain the cis-alkene targets, a Lindlar reduction of alkynes 40a and 40b provided both final products 41a and 41b. See FIG. 8. However, when attempting to use Birch-type conditions to give the trans-alkenes, only compound 39a underwent the reduction to give 42a.

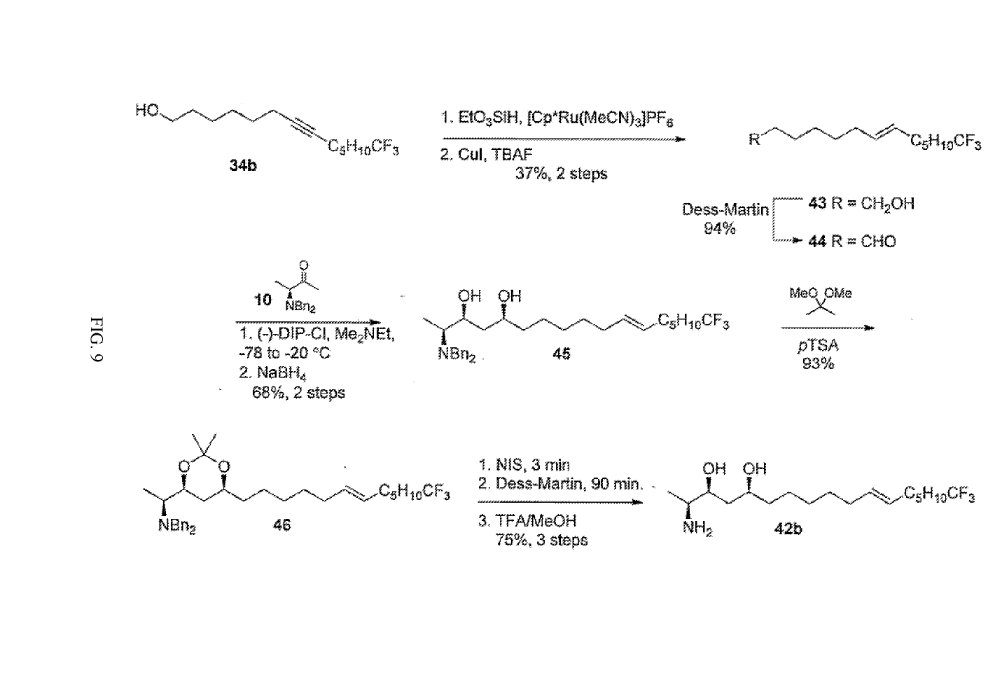

In order to attain the alkene 42b, the hydrosilation procedure was applied to the substrate 34b. See FIG. 9. Hydrosilation and protodesilylation did furnish the desired trans-alkene. The trans alcohol was carried through the same sequence as outlined previously to give the trans product 42b in 45% yield, an increase from the alkynyl compounds (18-21%).

### Infectious Diseases

In some embodiments, the disclosure relates to treating or preventing an infection by viruses, bacteria, fungi, protozoa, and parasites.

In one aspect of the disclosure, an “infection” or “viral infection” refers to an infection caused by adenovirus, coxsackievirus, hepatitis A virus, poliovirus, rhinovirus, herpes simplex, type 1, herpes simplex, type 2, varicella-zoster virus, epstein-barr virus, human cytomegalovirus, Human herpesvirus, type 8, hepatitis B virus, hepatitis C virus, yellow fever virus, dengue virus, west nile virus, human immunodeficiency virus (HIV), influenza virus, measles virus, mumps virus, parainfluenza virus, respiratory syncytial virus, human metapneumovirus, papillomavirus, rabies virus, rubella virus, human bocavirus, an parvovirus B19.

In one aspect of the disclosure, an “infection” or “bacterial infection” refers to an infection caused by acinetobacter spp, bacteroides spp, burkholderia spp, campylobacter spp, chlamydia spp, chlamydophila spp, clostridium spp, enterobacter spp, enterococcus spp, escherichia spp, fusobacterium spp, gardnerella spp, haemophilus spp, helicobacter spp, klebsiella spp, legionella spp, moraxella spp, morganella spp, mycoplasma spp, neisseria spp, peptococcus spp peptostreptococcus spp, proteus spp, pseudomonas spp, salmonella spp, serratia spp., staphylococcus spp, streptoccocus spp, stenotrophomonas spp, or ureaplasma spp.

In one aspect of the disclosure, an “infection” or “bacterial infection” refers to an infection caused by acinetobacter baumanii, acinetobacter haemolyticus, acinetobacter junii, acinetobacter johnsonii, acinetobacter Iwoffi, bacteroides bivius, bacteroides fragilis, burkholderia cepacia, campylobacter jejuni, chlamydia pneumoniae, chlamydia urealyticus, chlamydophila pneumoniae, clostridium difficile, enterobacter aerogenes, enterobacter cloacae, enterococcus faecalis, enterococcus faecium, escherichia coli, gardnerella vaginalis, haemophilus par influenzae, haemophilus influenzae, helicobacter pylori, klebsiella pneumoniae, legionella pneumophila, methicillin-resistant staphylococcus aureus, methicillin-susceptible staphylococcus aureus, moraxella catarrhalis, morganella morganii, mycoplasma pneumoniae, neisseria gonorrhoeae, penicillin-resistant streptococcus pneumoniae, penicillin-susceptible streptococcus pneumoniae, peptostreptococcus magnus, peptostreptococcus micros, peptostreptococcus anaerobius, peptostreptococcus asaccharolyticus, peptostreptococcus prevotii, peptostreptococcus tetradius, peptostreptococcus vaginalis, proteus mirabilis, pseudomonas aeruginosa, quino lone-resistant staphylococcus aureus, quinolone-resistant staphylococcus epidermis, salmonella typhi, salmonella paratyphi, salmonella enteritidis, salmonella typhimurium, serratia marcescens, staphylococcus aureus, staphylococcus epidermidis, staphylococcus saprophyticus, streptoccocus agalactiae, streptococcus pneumoniae, streptococcus pyogenes, stenotrophomonas maltophilia, ureaplasma urealyticum, vancomycin-resistant enterococcus faecium, vancomycin-resistant enterococcus faecalis, vancomycin-resistant staphylococcus aureus, vancomycin-resistant staphylococcus epidermis, mycobacterium tuberculosis, clostridium perfringens, klebsiella oxytoca, neisseria miningitidis, proteus vulgaris, or coagulase-negative staphylococcus (including staphylococcus lugdunensis, staphylococcus capitis, staphylococcus hominis, or staphylococcus saprophytic),

In one aspect of the disclosure “infection” or “bacterial infection” refers to aerobes, obligate anaerobes, facultative anaerobes, gram-positive bacteria, gram-negative bacteria, gram-variable bacteria, or atypical respiratory pathogens.

In some embodiments, the disclosure relates to treating a bacterial infection such as a gynecological infection, a respiratory tract infection (RTI), a sexually transmitted disease, or a urinary tract infection.

In some embodiments, the disclosure relates to treating a bacterial infection such as an infection caused by drug resistant bacteria.

In some embodiments, the disclosure relates to treating a bacterial infection such as community-acquired pneumoniae, hospital-acquired pneumoniae, skin & skin structure infections, gonococcal cervicitis, gonococcal urethritis, febrile neutropenia, osteomyelitis, endocarditis, urinary tract infections and infections caused by drug resistant bacteria such as penicillin-resistant streptococcus pneumoniae, methicillin-resistant staphylococcus aureus, methicillin-resistant staphylococcus epidermidis and vancomycin-resistant enterococci, syphilis, ventilator-associated pneumonia, intra-abdominal infections, gonorrhoeae, meningitis, tetanus, tuberculosis.

In some embodiments, the disclosure relates to treating a fungal infections such as infections caused by tinea versicolor, microsporum, trichophyton, and epidermophyton, candidiasis, cryptococcosis and aspergillosis.

In some embodiments, the disclosure relates to treating an infection caused by protozoa including, but not limited to, malaria, amoebiasis, giardiasis, toxoplasmosis, cryptosporidiosis, trichomoniasis, leishmaniasis, sleeping sickness, or dysentery.

### Malaria

Certain compounds disclosed herein are useful to prevent or treat an infection of a malarial parasite in a subject and/or for preventing, treating and/or alleviating complications and/or symptoms associated therewith and can then be used in the preparation of a medicament for the treatment and/or prevention of such disease. The malaria may be caused by Plasmodium falciparum, P. vivax, P. ovale, or P. malariae.

In one embodiment, the compound is administered after the subject has been exposed to the malaria parasite. In another embodiment, the compound of formula I is administered before the subject travels to a country where malaria is endemic.

The compounds or the above-mentioned pharmaceutical compositions may also be used in combination with one or more other therapeutically useful substances selected from the group comprising antimalarials like quinolines (quinine, chloroquine, amodiaquine, mefloquine, primaquine, tafenoquine); peroxide antimalarials (artemisinin, artemether, artesunate); pyrimethamine-sulfadoxine antimalarials (e.g. Fansidar); hydroxynaphtoquinones (e.g. atovaquone); acroline-type antimalarials (e.g. pyronaridine); and antiprotozoal agents such as ethylstibamine, hydroxystilbamidine, pentamidine, stilbamidine, quinapyramine, puromycine, propamidine, nifurtimox, melarsoprol, nimorazole, nifuroxime, aminitrozole and the like.

In an embodiment, compounds disclosed herein can be used in combination one additional drug selected from the group consisting of chloroquine, artemesin, qinghaosu, 8-aminoquinoline, amodiaquine, arteether, artemether, artemisinin, artesunate, artesunic acid, artelinic acid, atovoquone, azithromycine, biguanide, chloroquine phosphate, chlorproguanil, cycloguanil, dapsone, desbutyl halofantrine, desipramine, doxycycline, dihydrofolate reductase inhibitors, dipyridamole, halofantrine, haloperidol, hydroxychloroquine sulfate, imipramine, mefloquine, penfluridol, phospholipid inhibitors, primaquine, proguanil, pyrimethamine, pyronaridine, quinine, quinidine, quinacrineartemisinin, sulfonamides, sulfones, sulfadoxine, sulfalene, tafenoquine, tetracycline, tetrandine, triazine, salts or mixture thereof

### Cancer

In a typical embodiment, the disclosure relates to a method treating cancer comprising administering to a patient a pharmaceutical composition disclosed herein. In some embodiments, the disclosure relates to a compound of formula I, or a pharmaceutically acceptable salt thereof, as defined herein for use in the treatment of solid tumors such as carcinoma and sarcomas and the leukaemias and lymphoid malignancies.

In some embodiments, the disclosure relates to a compound of formula I, or a pharmaceutically acceptable salt thereof, as defined herein for use in the treatment of cancer of the breast, colorectum, lung (including small cell lung cancer, non-small cell lung cancer and bronchioalveolar cancer) and prostate.

In some embodiments, the disclosure relates to a compound of formula I, or a pharmaceutically acceptable salt thereof, as defined herein for use in the treatment of cancer of the bile duct, bone, bladder, head and neck, kidney, liver, gastrointestinal tissue, esophagus, ovary, pancreas, skin, testes, thyroid, uterus, cervix and vulva, and of leukaemias (including ALL and CML), multiple myeloma and lymphomas.

In some embodiments, the disclosure relates to a compound of formula I, or a pharmaceutically acceptable salt thereof, as defined herein for use in the treatment of cancer of the bile duct, bone, bladder, head and neck, kidney, liver, gastrointestinal tissue, esophagus, ovary, endometrium, pancreas, skin, testes, thyroid, uterus, cervix and vulva, and of leukaemias (including ALL and CML), multiple myeloma and lymphomas.

In some embodiments, the disclosure relates to a compound of formula I, or a pharmaceutically acceptable salt thereof, as defined herein for use in the treatment of lung cancer, prostate cancer, melanoma, ovarian cancer, breast cancer, endometrial cancer, kidney cancer, gastric cancer, sarcomas, head and neck cancers, tumors of the central nervous system and their metastases, and also for the treatment of glioblastomas.

In some embodiments, compounds disclosed herein could be used in the clinic either as a single agent by itself or in combination with other clinically relevant agents. This compound could also prevent the potential cancer resistance mechanisms that may arise due to mutations in a set of genes.

The anti-cancer treatment defined herein may be applied as a sole therapy or may involve, in addition to the compound of the disclosure, conventional surgery or radiotherapy or chemotherapy. Such chemotherapy may include one or more of the following categories of antitumour agents:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulfan and nitrosoureas); antimetabolites (for example antifolates such as fluoropyrimidines like 5-fluorouracil and gemcitabine, tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin); and proteosome inhibitors (for example bortezomib [Velcade®]); and the agent anegrilide [Agrylin®]; and the agent alpha-interferon;

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-erbb2 antibody trastuzumab [Herceptin™] and the anti-erbbl antibody cetuximab), farnesyl transferase inhibitors, tyrosine kinase inhibitors and serine/threonine kinase inhibitors, for example inhibitors of the epidermal growth factor family (for example EGFR family tyrosine kinase inhibitors such as: N-(3-chloro-4-fluorophenyl)-7-methoxy-6-(3-morpholinopropoxy)quinazolin-4-amine (gefltinib), N-(3-ethynylphenyl)-6,7-bis(2-methoxyethoxy)quinazolin-4-amine (erlotinib), and 6-acrylamido-N-(3-chloro-4-fluorophenyl)-7-(3-morpholinopropoxy)quinazolin-4-amine (CI 1033), for example inhibitors of the platelet-derived growth factor family and for example inhibitors of the hepatocyte growth factor family, for example inhibitors or phosphotidylinositol 3-kinase (PI3K) and for example inhibitors of mitogen activated protein kinase kinase (MEK1/2) and for example inhibitors of protein kinase B (PKB/Akt), for example inhibitors of Src tyrosine kinase family and/or Abelson (AbI) tyrosine kinase family such as dasatinib (BMS-354825) and imatinib mesylate (Gleevec™); and any agents that modify STAT signalling;

(v) antiangiogenic agents such as those which inhibit the effects of vascular endothelial growth factor, (for example the anti-vascular endothelial cell growth factor antibody bevacizumab [Avastin™]) and compounds that work by other mechanisms (for example linomide, inhibitors of integrin ocvβ3 function and angiostatin);

(vi) vascular damaging agents such as Combretastatin A4;

(vii) antisense therapies, for example those which are directed to the targets listed above, such as an anti-ras antisense;

(viii) gene therapy approaches, including for example approaches to replace aberrant genes such as aberrant p53 or aberrant BRCA1 or BRCA2, GDEPT (gene-directed enzyme prodrug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies, and approaches using the immunomodulatory drugs thalidomide and lenalidomide [Revlimid®].

Such conjoint treatment may be achieved by way of the simultaneous, sequential or separate dosing of the individual components of the treatment. Such combination products employ the compounds of this disclosure, or pharmaceutically acceptable salts thereof, within the dosage range described hereinbefore and the other pharmaceutically-active agent within its approved dosage range.

The disclosure relates to phosphorylation inhibitors of PEVI kinases. In still further embodiments, the disclosure relates to pharmaceutical composition comprising compounds disclosed herein and their use in the prevention and treatment of cancer.

### Formulations

The compounds of the disclosure may be in the form of pharmaceutically acceptable salts, as generally described below. Some preferred, but non-limiting examples of suitable pharmaceutically acceptable organic and/or inorganic acids are as hydrochloric acid, hydrobromic acid, sulfuric acid, nitric acid, acetic acid and citric acid, as well as other pharmaceutically acceptable acids known per se (for which reference is made to the prior art referred to below).

When the compounds of the disclosure contain an acidic group as well as a basic group the compounds of the disclosure may also form internal salts, and such compounds are within the scope of the disclosure. When the compounds of the disclosure contain a hydrogen-donating heteroatom (e.g. NH), the disclosure also covers salts and/or isomers formed by transfer of said hydrogen atom to a basic group or atom within the molecule.

Pharmaceutically acceptable salts of the compounds of formula I include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein may be administered in the form of prodrugs. A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds according to formula I.

Pharmaceutical composition for use in the present disclosure typically comprises an effective amount of a compound of formula I and a suitable pharmaceutical acceptable carrier.

The preparations may be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is again made to U.S. Pat. No. 6,372,778, U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

Generally, for pharmaceutical use, the compounds may be formulated as a pharmaceutical preparation comprising at least one compound of formula I and at least one pharmaceutically acceptable carrier, diluent or excipient and/or adjuvant, and optionally one or more further pharmaceutically active compounds.

The pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the disclosure, e.g. about 10, 25, 50, 100, 200, 300 or 400 mg per unit dosage.

The compounds can be administered by a variety of routes including the oral, ocular, rectal, transdermal, subcutaneous, intravenous, intramuscular or intranasal routes, depending mainly on the specific preparation used. The compound of formula I will generally be administered in an “effective amount”, by which is meant any amount of a compound of the Formula I that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which may be administered as a single daily dose, divided over one or more daily doses. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. No. 6,372,778, U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

For an oral administration form, the compound of formula I can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, the compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the disclosure or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant.

For subcutaneous or intravenous administration, the compounds of formula I, if desired with the substances customary therefore such as solubilizers, emulsifiers or further auxiliaries are brought into solution, suspension, or emulsion. The compounds of formula I can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, the formulations may be prepared by mixing the compounds of formula I with a suitable non-irritating excipient, such as cocoa butter, synthetic glyceride esters or polyethylene glycols, which are solid at ordinary temperatures, but liquefy and/or dissolve in the rectal cavity to release the drug.

The compounds may be formulated for local effect, for instance topical or non-adsorbent applications.

### EXPERIMENTAL

### Example 1

### In Vitro Susceptibility

P. falciparum:

Two strains of P. falciparum were tested, namely W2, known as a chloroquine resistant strain, and D6, a chloroquine sensitive strain. Cryopreserved iRBCs were provided by Dr. John W. Barnwell from the Centers for Disease Control and Prevention and maintained in in vitro culture as described in Trager & Jensen, Science 1976, 193 (4254), 673-5.

P. knowlesi:

The H strain of P. knowlesi has been adapted to in vitro culture using Macaca mulatta (rhesus monkey) RBCs essentially as described in Kocken et al., Infect Immun 2002, 70 (2), 655-60 (the media was supplemented with 10% human AB serum).

Compounds:

Chloroquine (Sigma) and mefloquine (Sigma) were used as controls. Compounds in Table 1 were dissolved in 70% 200-proof pharmaceutical grade ethanol, and a working solution of 0.2 mg/mL was prepared using hypoxanthine- and gentamicin-free incomplete RPMI 1640 (Gibco). Compounds in Table 2 were dissolved in 200 proof pharmaceutical grade ethanol at a concentration of 50 μM and then conjugated to BSA (Sigma) as described.⁵ A working solution of 0.2 mg/mL was prepared using hypoxanthine- and gentamicin-free incomplete RPMI 1640 (Gibco). Two compounds were tested per plate in triplicate starting at 0.2 mg/mL and diluted two-fold down to 0.019 μg/mL using hypoxanthine- and gentamicin-free RPMI.

Incorporation Assay:

Parasite inoculum was prepared by mixing parasitized blood, uninfected 0+RBCs (for P. falciparum strains) or rhesus RBCs (for P. knowlesi) and culture medium (deficient in hypoxanthine and gentamicin) to a final concentration of 1% parasitemia and 1% haematocrit. A negative control was prepared by diluting uninfected erythrocytes to 1% haematocrit in culture medium. [³H]-labeled hypoxanthine was added at 24 h (for P. falciparum strains) or 12 h (P. knowlesi) and the culture was collected 48 h or 12 h later for P. falciparum or P. knowlesi, respectively. [³H] uptake was measured by using a scintillation spectrophotometer (Wallac Oy 1450 MicroBeta reader).

Data Analyses:

Triplicate wells were used to calculate the IC₅₀ for each compound using the software XLFit v5.2 using the sigmoidal model 601. Average and standard deviations were calculated using Microsoft Excel.

### Example 2

### Morphological Changes Upon Incubation with N-Methylenigmol (ESPD 507)

A dilution of 1% P. falciparum W2 parasites (82% ring stage) was prepared as described above and incubated with 546.94 ng/mL (IC₅₀=1.73 μM) of the test analog for 48 h. Samples were removed every 4 h and thin blood smears were prepared, stained with modified Giemsa stain, and analyzed by light microscopy for morphological changes in growth and development. The modular incubator used was gassed and returned to 37° C. in less than 5 minutes to avoid delay in parasite growth.

### Example 3

### Mouse PK Studies

Enigmol (E) or N-Methyl-enigmol (NME) were dissolved as 10× stocks in ethanol, then diluted 1:10 in olive oil the day before dosing; dosing solutions were stored at room temperature overnight. Male mice, CD-1 or Swiss Webster (20 to 30 g), were dosed p.o. at either 10 or 30 mg/kg and 5 ml/kg (n=3/time point). For single dose studies with E and NME, bloods were sampled at 0.5, 1, 2, 4, 6, 8, 16 & 24 hrs. For the 5 day repeat dose study with E, samples were taken on day 5 at 0 (trough) 0.5, 1, 2, 4, 6, 8 & 24 hrs. Blood (˜0.3 ml) was obtained either from the submandibular vein using a 4 mm Small Animal Lancet (MEDIpoint), or by retro-orbital bleeding into 200 uL glass micropipettes (Drummond) under isoflurane anesthesia. Each mouse was sampled once and blood transferred immediately to 0.5 ml K EDTA or Li heparin microtainers (BD) in ice water. Tubes were centrifuged at 2000×g for 10 min in a refrigerated centrifuge to separate plasma from RBCs. The plasma supernatants and RBC pellets (when taken) were transferred into separate 1.5 mL Eppendorf tubes in ice water, then frozen on dry ice and stored at −80° C. prior to analysis by LC/MS/MS.

### Example 4

### Bioanalytical Assay Method and Pharmacokinetic Analysis

Mouse plasma determinations of Enigmol and N-methylenigmol were done using a bioanalytical assay method consisting of a protein precipitation sample preparation step followed by analysis on an AB SCIEX QTRAP® 5500 LC/MS/MS System. The method employed an internal standard (ISTD) spiking technique with D17:0 sphinganine as the ISTD. The lower limit of quantitation (LLOQ) for the assay method was 10 ng/mL and the upper limit of quantitation (ULOQ) for the assay method was 1,000 ng/mL for both Enigmol and N-methylenigmol. Each bioanalytical run consisted of eight matrixes (mouse plasma) spiked calibration standards and six matrixes spiked quality control (QC) samples at three levels (30 ng/mL, 500 ng/mL, and 900 ng/mL) in duplicate along with unknown mouse study samples. Enigmol and N-methylenigmol mean precision (% CV) based upon QC sample results during sample analysis was 9% and 10%, respectively. Enigmol and N-methylenigmol mean accuracy (% DEV) based upon QC sample results during sample analysis was 9% and 6%, respectively. Estimates of pharmacokinetic parameters from the single dose (10 mg/kg and 30 mg/kg PO) mouse studies were determined using WinNonlin® (Pharsight, Version 5.3).

### Example 5

### Rat Tissue Distribution

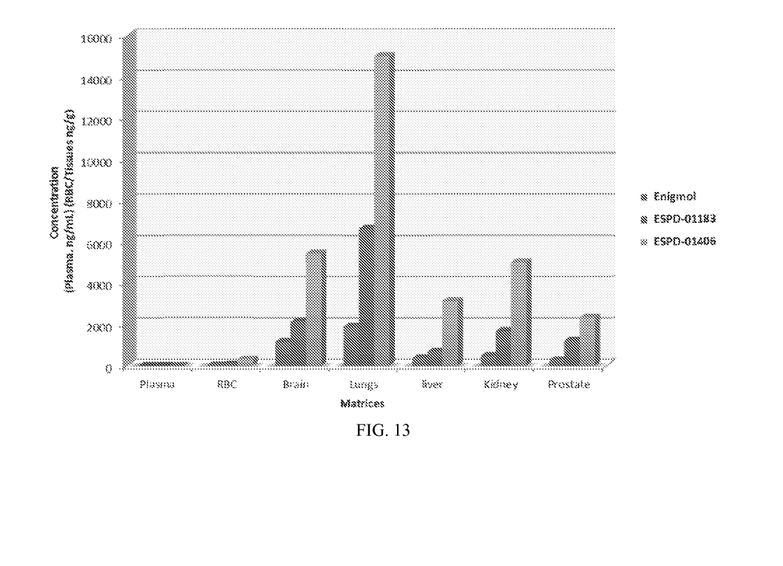

Pre-cannulated male SD rats were given a 10 mg/kg dose of drug p.o. using a PEG 400/Tween 80 formulation, and were sacrificed with CO2 after 24 hrs. Selected tissue and organs were harvested, homogenized, and analyzed via LC/MS/MS for tissue/organ-drug concentration. The results for Enigmol, ESPD-01183 and ESPD-01406 are provided in FIG. 13.

### Example 6

### Mouse Tumor Concentrations

Nude mice were subjected to human prostate cancer xenograft procedures, and resulting tumors were allowed to grow for 16 days. On day 17, mice began a drug treatment schedule of 10 or 30 mg/kg p.o. once daily ending on day 38. Once the scheduled time course had passed, the mice were sacrificed. Tumors were harvested, homogenized, and analyzed by LC/MS/MS for tumor-drug concentration. The results are provided in the table below.

| | 3 mg/kg | 10 mg/kg | 3 mg/kg | 10 mg/kg |
|---|---|---|---|---|
| Control | Enigmol | Enigmol | CF3-Enigmol | CF3-Enigmol |
| 0 | 317 | 1565 | 1147 | 9004 |

### Example 7

### Mouse Xenograft Cancer Model

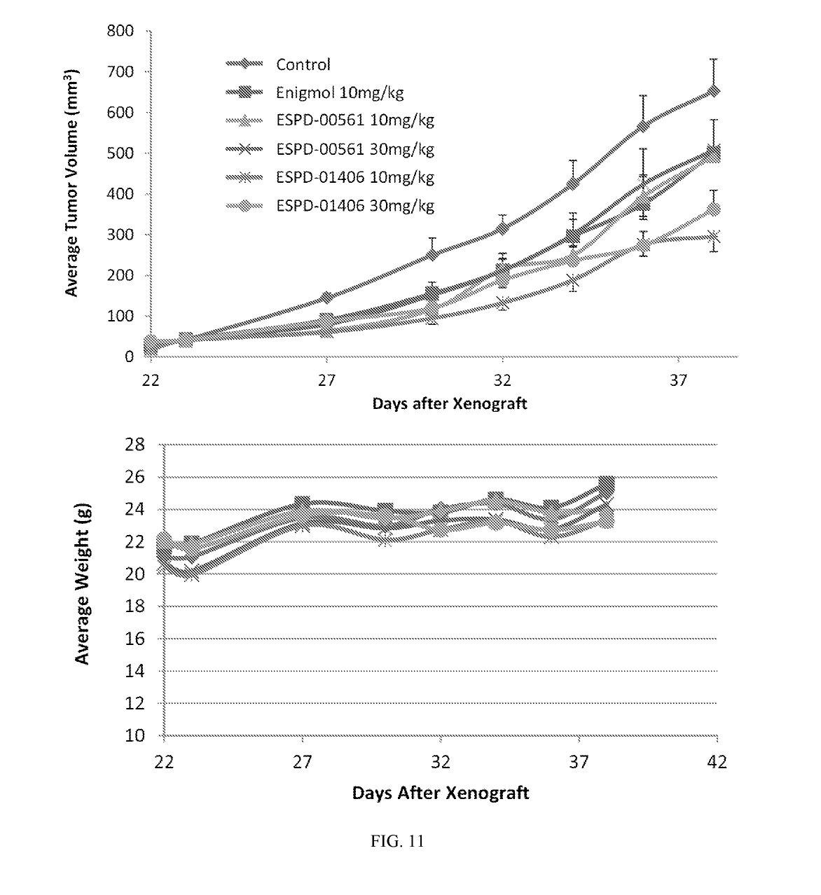

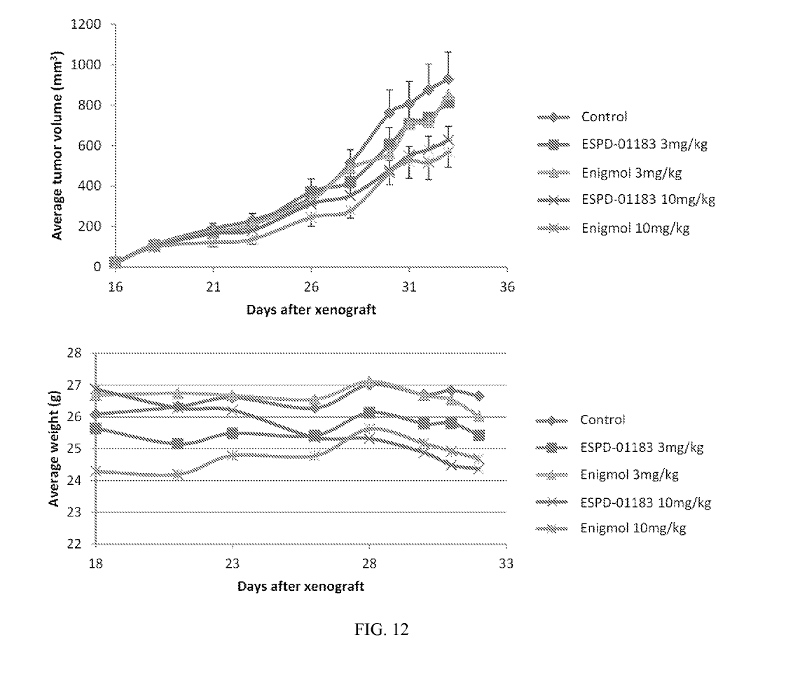

Nude mice (n=10/group) were subjected to human prostate cancer xenograft procedures, and resulting tumors were allowed to grow for 16 or 22 days. From day 17 to day 33 or from day 23 to day 38, mice were subjected to a drug treatment schedule of 10 or 30 mg/kg p.o. once daily. Tumor volumes and body weights were measured periodically. Results are provided in FIGS. 11 and 12.

### Example 8

### Synthesis of Sphingolipid Analogs

Starting materials were synthesized via literature procedures. See FIG. 5 and Tanemura et al., Chemical Communications 2004, (4), 470-471; Bushnev et al., ARKIVOC 2010, 8, 263-277; and Krimen, Org. Synthesis 1970, 50. The syntheses of ESPD-0500, ESPD-0503, ESPD-0505, and ESPD-0507 as well as ESPD-0522 were prepared according to Garnier-Amblard et al., ACS Med. Chem. Letters 2011, 2, 438-443 and Moore, R. Part 1: Synthesis and biological evaluation of novel phenyl epothilone analog. Part 2: Oxazolidinone directed diastereoselective synthesis of beta-amino carbonyl derivatives. Part 3: Synthesis of novel sphingolipid analogs. Emory University, Atlanta, 2006.

### Preparation of 2-bromo-5-hydroxy-2-methyloctadecan-3-one (16)

(−)-DIP-Cl (chlorobis((2S,3R)-3,6,6-trimethylbicyclo[3.1.1]heptan-2-yl)borane) (55% wt in THF, 21 mL, 33 mmol) and dimethylethylamine (9.8 mL, 91 mmol) was dissolved in dry THF (100 mL) and cooled to 0° C. 3-Bromo-3-methylbutan-2-one (5.0 g, 30 mmol) was added dropwise and let stir at 0° C. for 30 min. The solution became cloudy and slightly pink. It was cooled the reaction to −78° C. and tetradecanal (7.1 g, 33 mmol) was added. The solution was stirred at −78° C. for 3 hrs; warmed to ambient temperature; cooled back to −78° C.; 50 mL of MeOH and 20 mL of 30% H₂O₂ was added slowly (exothermic); and let stir for 3 hrs more warming to ambient temperature. Water was added, and the product was extracted with ether (3×500 mL). The combined organics were washed with brine, dried over magnesium sulfate, and concentrated to give a colorless viscous oil. The product was purified via automated flash chromatography (silica, 0-25% EtOAc:Hex) to give a waxy solid. 6.38 g (56%). Subsequent mosher ester analysis showed a racemic mixture of products. Rf=0.3 (20% EtOAc:Hex); mp: ˜30° C.; ¹H NMR [600 MHz, CDCl₃] δ 4.06 (br s, 1H), 3.04 (dd, 1H, J=2.4, 17.4 Hz), 2.91 (br s, 1H), 2.87 (dd, 1H, J=9.0, 17.4 Hz), 1.86 (d, 6H, J=9.6 Hz), 1.57-1.53 (m, 1H), 1.47-1.43 (m, 2H), 1.40-1.24 (m, 21H), 0.88 (t, 3H, J=7.0 Hz); ¹³C NMR [100 MHz, CDCl₃] δ 206.9, 68.49, 68.46, 63.8, 43.1, 36.7, 32.1, 29.9, 29.79, 29.76, 29.6, 29.5, 29.4, 25.7, 22.9, 14.3 ppm; HRMS (ESI) m/z 377.20548 (Theo. for C₁₉H₃₇BrO₂+H: 377.20497); IR (ATR) 2921, 2852, 1710, 1627, 1459, 1370, 1109, 1077, 997, 721 cm⁻¹;

### Preparation of (S)-2-amino-5-hydroxy-2-methyloctadecan-3-one (ESPD-0562)

(S)-2-Bromo-5-hydroxy-2-methyloctadecan-3-one (16) (0.372 g, 0.99 mmol) and sodium azide (0.481 g, 7.4 mmol) was dissolved in a 1:1 mixture of methanol (7.5 mL) and water (7.5 mL) in 20 mL a microwave vial. The solution was heated via microwave at 105° C. for 1 hr. LCMS analysis showed the presence of the azide intermediate (m/z 362, M+Na). The resulting reaction mixture was extracted with ether (3×10 mL). The combined organics were dried over magnesium sulfate, and concentrated to give a white semi-solid. The solid was dissolved into 10 mL of methanol and 10% palladium on carbon (0.105 g, 0.01 mmol) was added. The solutions was stirred under hydrogen atmosphere (balloon) for 24 hrs. The resulting reaction mixture was filtered through a pad of celite and concentrated to give a white semi-solid. The produce was purified via automated flash chromatography (100% CHCl₃ for 3 Column volumes then 10% MeOH in CDCl₃ (w/ 1% NH₄OH) until elution of the desired peak, R_{f}=0.5 in 84:15:1 CHCl₃:MeOH:NH₄OH) to give a slightly yellow solid (0.155 g, 50%). mp 31-33° C.; ¹H NMR [400 MHz, CDCl₃] δ 4.03-3.97 (m, 1H), 2.69 (d, 2H, J=6.0 Hz), 2.45-2.20 (br s, 3H), 1.56-1.38 (m, 4H), 1.32-1.24 (m, 26H), 0.88 (t, 3H, J=6.8 Hz); ¹³C NMR [100 MHz, CDCl₃] δ 216.1, 68.0, 59.3, 43.7, 37.0, 32.1, 29.80 (m), 29.75 (m), 29.5, 27.3, 27.2, 25.7, 22.8, 14.3 ppm; HRMS [ESI] m/z 316.30535 (calc'd for C₁₉H₃₉NO₂+H: 316.30536); IR (ATR) 3379 (br), 2954, 2915, 2848, 1693, 1470, 1365, 1127, 1099, 1049, 1022, 984, 843, 718, 599 cm⁻¹; Elem. Anal. C, 72.79; H, 12.77; N, 4.40 (calc'd for C₁₉H₃₉NO₂: C, 72.79; H, 12.54; N, 4.47).

### Preparation of (+/−)-syn-2 amino-2-methyloctadecane-3,5-diol (ESPD-0560) and (+/−)-anti-2 amino-2-methyloctadecane-3,5-diol (ESPD-0561)

2-Amino-5-hydroxy-2-methyl-octadecan-3-one (ESPD-0562) (0.543 g, 1.7 mmol) was dissolved in dry methanol (20 mL) and cooled to −40° C. Sodium borohydride (98 mg, 2.6 mmol) was added slowly in portions (5×˜20 mg) and let stir for 2.5 hrs at −40° C. Saturated sodium bicarbonate was added, and the product was extracted with DCM (3×10 mL). The combined organics were dried over magnesium sulfate and concentrated to give a clear gel. The composition was purified via chromatography (100% CHCl₃ to 89:10:1-CHCl₃:MeOH:NH₄OH) to give the syn diol product (227 mg, 45%) as a clear oil which solidified upon standing as well as the anti diol (92 mg, 17%) as a semisolid oil. The relative stereochemistry of the 1,3-diol was determined using Rychnovsky's ¹³C acetonide method. The identifying the syn diol with acetonide carbon resonances of 98.6 ppm, 30.4, and 20.0 ppm and the anti diol with resonances of 100.4 ppm and 24.8, 24.5 ppm.

Syn diol (ESPD-0560): R_{f}=0.2 (84:15:1-CHCl₃:MeOH:NH₄OH); mp 46-51° C.; ¹H NMR [400 MHz, CDCl₃] δ 3.88-3.78 (m, 1H), 3.49 (d, 1H, J=9.2 Hz), 2.83 (br s, 4H), 1.61 (d, 1H), 1.55-1.22 (m, 26H), 1.12 (s, 3H), 1.06 (s, 3H), 0.88 (t, 3H, J=6.6 Hz); ¹³C NMR [100 MHz, CDCl₃] δ 79.2, 72.4, 52.9, 38.4, 37.4, 32.1, 29.95, 29.90 (m), 29.87 (m), 29.6, 28.4, 25.8, 24.8, 22.9, 14.3 ppm; HRMS [APCI] m/z 316.32115 (calc'd for C₁₉H₄₁NO₂+H: 316.32101); IR (ATR) 3343, 2916, 2849, 1588, 1465, 1368, 1217, 1084, 1023, 985, 893, 846, 720 cm⁻¹.

Anti diol (ESPD-0561): R_{f}=0.15 (84:15:1-CHCl₃:MeOH:NH₄OH); ¹H NMR [400 MHz, CDCl₃] δ 3.91-3.84 (m, 1H), 3.58 (dd, 1H, J=6.8, 6.4 Hz), 2.90-2.20 (br s, 4H), 1.58-1.40 (m, 4H), 1.35-1.24 (m, 20H), 1.11 (s, 3H), 1.06 (s, 3H), 0.88 (t, 3H, J=6.4 Hz); ¹³C NMR [100 MHz. CDCl₃] δ 74.7, 69.2, 52.9, 37.9, 37.7, 32.1, 29.89 (m), 29.85 (m), 29.6, 28.1, 26.2, 25.0, 22.9, 14.3 ppm; HRMS [ESI] m/z 316.32114 (calc'd for C₁₉H₄₁NO₂+H: 316.32101); IR (ATR) 3262, 2916, 2849, 1597, 1467, 1378, 1069, 719, 635 cm⁻¹.

### Preparation of (+/−)-anti-2 amino-2-methyloctadecane-3,5-diol (ESPD-0561)

Anti 1,3-diol could be generated independently. A mixture of (S)-2-amino-5-hydroxy-2-methyloctadecan-3-one (100 mg, 0.32 mmol) in 5.0 mL of dry methanol was cooled to −40° C. Sodium triacetoxyborohydride (101 mg, 0.48 mmol) was added slowly in portions (5×˜20 mg); let stir at −40° C. for 2.5 hrs; and then warmed to ambient temperature. Removal of the solvent gave a white solid which was purified via chromatography (isocratic 84:15:1-CHCl₃:MeOH:NH₄OH) providing a clear oil that semisolidified upon standing. (95 mg, 94%).

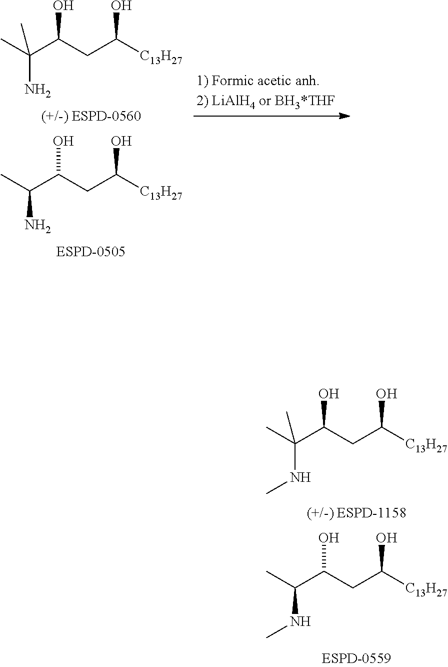

### Preparation of (2S,3R,5S)-2-(methylamino)octadecane-3,5-diol (ESPD-0559)

(2S,3R,5S)-2-Aminooctadecane-3,5-diol (ESPD-0505) (100 mg, 0.33 mmol) was dissolved in dry dioxane (5 mL) and cooled to 0° C. Acetic formic anhydride (32 mg, 0.37 mmol) was added dropwise, and the resulting reaction mixture was stirred for 1 hr warming to room temperature. The volatiles were removed under reduced pressure and the formate intermediate was isolated via chromatography (0-10% MeOH:DCM) to give 95 mg of a white solid. The resulting solid was dissolved in dry THF (5 mL), and lithium aluminium hydride (1M in THF, 1.0 ml, 1.0 mmol) was added dropwise and then heated to 60° C. overnight. The reaction was cooled to ambient temperature; 1 mL of 1 M NaOH was added; and the organics separated. The aqueous was washed with DCM several times and the organics were combined dried over magnesium sulfate and concentrated. The resulting material was purified via chromatography (100% CHCl₃ to 89:10:1-CHCl₃:MeOH:NH₄OH) to give 12 mg (11%) of a white solid. [α]²⁰_{D}=+17° (c=0.25, CHCl₃); ¹H NMR [600 MHz, CDCl₃] δ 3.93-3.89 (m, 1H), 3.88-3.84 (m, 1H), 2.79 (br s, 3H), 2.57-2.52 (m, 1H), 2.44 (s, 3H), 1.63 (ddd, 1H, J=13.8, 9.9, 2.7 Hz), 1.54-1.42 (m, 4H), 1.38 (ddd, 1H, 13.8, 8.7, 2.7 Hz), 1.34-1.22 (m, 20H), 1.03, (d, 3H, 6.0 Hz), 0.88 (t, 3H, J=7.2 Hz); ¹³C NMR [150 MHz, CDCl₃] δ 69.2, 68.8, 59.6, 39.8, 37.8, 34.1, 32.1, 29.90 (m), 29.86 (m), 29.6, 26.2, 22.9, 14.3 ppm; HRMS [ESI] m/z 316.32018 (calc'd for C₁₉H₄₁NO₂+H: 316.32101); IR (ATR) 3360, 3281, 2919, 2849, 1467, 1378, 1084, 1039, 802, 721, 670 cm⁻¹.

### Preparation of (+/−)-syn-2-methyl-2-(methylamino)octadecane-3,5-diol (ESPD-1158)

(+/−)-Syn-2-amino-2-methyloctadecane-3,5-diol (ESPD-0560) (203 mg, 0.64 mmol) was dissolved in dry DCM (10 ml), and acetic formic anhydride (113 mg, 1.3 mmol) was added dropwise. The resulting reaction mixture was stirred for 20 min. The solvent was removed under reduce pressure to give the crude formamide intermediate as a white solid. The product was purified via chromatography (1-10% MeOH:DCM) to give a clear oil (199 mg). The intermediate was dissolved in 7.5 mL of dry THF, and borane*THF complex (2.57 ml, 2.6 mmol) was added. The solutions was let stir until bubbling subsided. The resulting solution was heated at 140 degrees ° C. for 40 min via microwave irradiation. 12 Drops of 12M HCl was added to the reaction vial and let stir (bubbling) at RT for 30 min. This was poured into 50 mL of 1M NaOH (pH after combination was ˜13) and extracted with ethyl acetate (3×5 mL). The resulting organics were combined, washed with brine, dried over magnesium sulfate, and concentrated to give a clear oil. The product was purified via chromatography (99:1:0.1-DCM:MeOH:NH₄OH to 89:10:1-DCM:MeOH:NH₄OH) to give a clear oil that solidified to a white solid under vacuum overnight. (86 mg, 40%).; ¹H NMR [400 MHz, CDCl₃] δ 3.86-3.78 (m, 1H), 3.55 (dd, 1H, J=9.8, 2.0 Hz), 2.31 (s, 3H), 1.58-1.37 (m, 6H), 1.33-1.24 (m, 20H), 1.07 (s, 3H), 0.99 (s, 3H), 0.88 (t, 3H, J=6.8 Hz); ¹³C NMR [150 MHz, CDCl₃] δ 76.5, 72.1, 56.5, 38.2, 37.5, 32.1, 30.0, 29.89, 29.86, 29.6, 28.0, 25.8, 22.9, 22.2, 20.9, 14.3 ppm; HRMS [ESI] m/z 330.33638 (calc'd for C₂₀H₄₃NO₂+H: 330.33666); IR (ATR) 3297, 2917, 2850, 1469, 1373, 1072, 910, 855, 843, 720, 611 cm⁻¹.

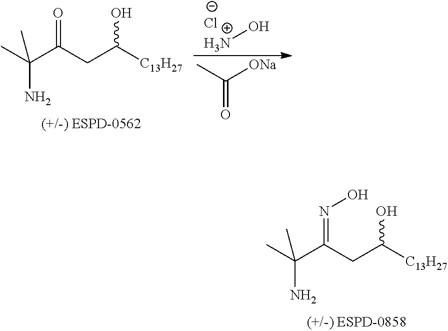

### Preparation of (+/−,E)-2-amino-5-hydroxy-2-methyloctadecan-3-one oxime (ESPD-0858)

2-Amino-5-hydroxy-2-methyloctadecan-3-one (ESPD-0562) (50 mg, 0.16 mmol) was dissolved in 2.5 mL of dry methanol, and a mixture of sodium acetate (39 mg, 0.48 mmol) and hydroxylamine hydrochloride (22 mg, 0.32 mmol) dissolved in 2.5 mL of dry methanol was added. The solution was stirred at 60° C. overnight. The solvent was removed under reduced pressure and purified via chromatography (100% CHCl₃ to 89:10:1-CHCl₃:MeOH:NH₄OH) to give 16 mg (30%) of a white solid. ¹H NMR [400 MHz, CDCl₃] δ 3.88-3.83 (m, 1H), 3.49 (s, 1H), 2.90 (dd, 1H, J=13.4, 1.6 Hz), 2.29 (dd, 1H, J=13.4, 9.2 Hz), 1.57-1.42 (m, 6H), 1.42 (s, 3H), 1.33 (s, 3H), 1.33-1.24 (m, 21H), 0.88 (t, 3H, J=6.8 Hz); ¹³C NMR [100 MHz, CDCl₃] 6 163.4, 69.3, 53.9, 38.7, 33.4, 32.1, 29.9 (m), 29.6, 29.0, 26.0, 22.9, 14.3 ppm; HRMS [ESI] m/z 329.31643 (calc'd for C₁₉H₄₀N₂O₂+H: 329.31626); IR (ATR) 3191, 3066, 2918, 2850, 1514, 1468, 1367, 1184, 1124, 1023, 982, 955, 719 cm⁻¹.

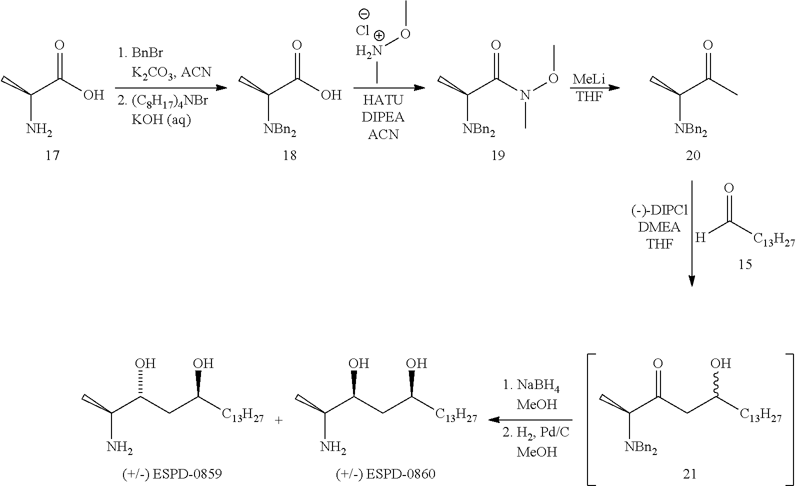

### Preparation of 1-(dibenzylamino)-N-methoxy-N-methylcyclopropanecarboxamide (19)

1-(Dibenzylamino)cyclopropanecarboxylic acid (18) (505 mg, 1.8 mmol) and HATU (2-(7-aza-1H-benzotriazole-1-yl)-1,1,3,3-tetramethyluronium hexafluorophosphate) (687 mg, 1.8 mmol) was dissolved in acetonitrile (15.0 mL), stirred 5 min, then N,O-dimethylhydroxylamine hydrochloride (438 mg, 4.5 mmol) followed by diisopropylethylamine (1.56 mL, 9.0 mmol) was added. The mixture was heated with microwave irradiation to 100° C. for 50 min; cooled to ambient temperature; and the solvent was removed under reduced pressure. The resulting residue was purified via chromatography (0-50% EtOAc:Hex) to give 491 mg (84%) of a clear oil. ¹H NMR [400 MHz, CDCl₃] δ 7.26-7.15 (m, 10H), 3.93 (s, 4H), 3.60 (s, 3H), 3.24 (s, 3H), 1.50 (dd, 2H, J=7.2, 4.8 Hz), 0.87 (dd, 2H, J=7.2, 4.8 Hz); ¹³C NMR [150 MHz, CDCl₃] δ 172.6, 140.5, 129.2, 128.1, 126.9, 61.0, 57.3, 46.8, 34.4, 15.3 ppm; HRMS [ESI] m/z 325.19090 (calc'd for C₂₀H₂₄N₂O₂+H: 325.19105); IR (ATR) 3026, 2935, 1638, 1493, 1452, 1410, 1364, 1136, 1075, 1027, 997, 747, 729, 696, 513 cm⁻¹.

### Preparation of 1-(1-(dibenzylamino)cyclopropyl)ethanone (20)

1-(Dibenzylamino)-N-methoxy-N-methylcyclopropanecarboxamide (19) (1.92 g, 5.9 mmol) in tetrahydrofuran (40 ml) were dissolved and cooled to −78° C. Methyllithium (1.25 M in THF, 13.6 ml, 17 mmol) was added slowly over a period of 1 hr via syringe pump, and stirred for a further 30 min. The reaction was very slowly quenched with 50 mL of sat. ammonium chloride, allowed to warm to room temperature, and extracted with ether. The organics were dried over magnesium sulfate and concentrated to yield 1.56 g (94%) of a yellow oil, which did not require further purification. ¹H NMR [400 MHz, CDCl₃] δ 7.27-7.17 (m, 10H), 4.00 (s, 4H), 1.85 (s, 3H), 1.14 (dd, 2H, J=7.6, 4.8 Hz), 0.91 (dd, 2H, J=7.6, 4.8 Hz); ¹³C NMR [100 MHz, CDCl₃] δ 209.8, 140.4, 129.0, 128.2, 126.9, 57.2, 53.1, 26.0, 19.4 ppm; HRMS [ESI] m/z 280.16941 (calc'd for C₁₉H₂₁NO+H: 280.16959); IR (ATR) 3025, 2848, 1586, 1493, 1452, 1354, 1305, 1266, 1129, 1027, 854, 821, 741, 696, 524 cm⁻¹.

### Preparation of 1-(1-aminocyclopropyl)-3-hydroxyhexadecan-1-one (21)

(−)-DIP-Cl (chlorobis((2S,3R)-3,6,6-trimethylbicyclo[3.1.1]heptan-2-yl)borane) (3.9 ml, 6.1 mmol) and dimethylethylamine (1.95 ml, 16.7 mmol) were dissolved in dry THF (15 mL) and cooled to 0° C. 1-(1-(Dibenzylamino)cyclopropyl)ethanone (20) (1.56 g, 5.6 mmol), dissolved in 10 mL of dry THF was added dropwise and stirred at 0° C. for 30 min. The solution became cloudy and slightly pink. The reaction to −78° C. was cooled and tetradecanal (1.78 g, 8.4 mmol) dissolved in 10 mL of dry THF was added dropwise. This solution was stirred at −78° C. for 3 hrs; warmed to RT over the last hour; cooled again to −78° C.; added 15 mL of MeOH and 10 mL of 30% H₂O₂; let stir for 3 hrs; and warmed to RT overnight. Water was added and the product was extracted with ether (3×30 mL). The combined organics were washed with brine, dried over magnesium sulfate, and concentrated to give a clear oil The product was purified via chromoatography (0-20% EtOAc:Hex) to yield 1.10 g (40%) of a clear oil which was ˜77% pure with starting material as the contaminant. The product was found to be relatively unstable with the appearance of starting material (20) over time presumably due to retro aldol. Therefore it was immediately reduced to the diol without further purification or characterization. R_{f}=0.2 (10% EtOAc:Hex, H₂SO₄ stain); ¹H NMR [400 MHz, CDCl₃] δ 7.30-7.21 (m, 10H), 4.02 (s, 4H), 4.02-3.95 (m, 1H), 3.46 (br s, 1H), 2.40 (dd, 1H, J=17.2, 2.8 Hz), 2.15 (dd, 1H, J=17.2, 8.4 Hz), 1.55-1.40 (m, 3H), 1.40-1.26 (m, 22H), 1.18-1.15 (m, 1H), 1.03-0.96 (m, 2H), 0.92 (t, 3H, J=6.8 Hz); ¹³C NMR [100 MHz, CDCl₃] δ 213.4, 140.0, 129.0, 128.2, 127.0, 68.0, 57.1, 53.1, 44.2, 36.7, 32.0, 31.7, 29.8 (m), 29.5, 25.6, 22.8, 19.8, 19.1, 14.3 ppm; LCMS [ESI] m/z 492.4 (calc'd for C₃₃H₄₉NO₂+H: 492.4).

### Preparation of (+/−)-anti-1-(1-aminocyclopropyl)hexadecane-1,3-diol (ESPD-0859) and (+/−)-syn1-(1-aminocyclopropyl)hexadecane-1,3-diol (ESPD-0860)

(S)-1-(1-(Dibenzylamino)cyclopropyl)-3-hydroxyhexadecan-1-one (21) (367 mg, 0.75 mmol) was dissolved in dry methanol (8 mL) cooled to 0° C. Sodium borohydride (42 mg, 1.1 mmol) was added, and the solution was stirred under argon at 0° C. for 3 hrs. Saturated sodium bicarbonate was added, and the product was extracted with DCM (3×10 mL). The combined organics were dried over magnesium sulfate and concentrated to give a clear oil. Purification of the product via chromatography could not achieve sufficient separation. The recovered mixture was dissolved in MeOH 15 mL and 250 mg of 10% Pd on carbon was added. The solution was agitated on a Parr shaker under 40 psi of hydrogen for 18 hrs. The solution was filtered through a pad of celite, and the solvent was removed to give a clear oil. The product was purified via chromatography (100% CHCl₃ to 89:10:1-CHCl₃:MeOH:NH₄OH) to give 35 mg of the anti diol (ESPD-0859) and 64 mg of the syn diol (ESPD-0860), both as clear oils. The relative stereochemistry of the 1,3-diol was determined using Rychnovsky's ¹³C acetonide method. The syn diol was identified with acetonide carbon resonances of 98.56 ppm, 30.39, and 20.02 ppm and the anti diol with resonances of 100.44 ppm and 24.80, 24.49 ppm.

Syn diol (ESPD-0860): R_{f}=0.2 (84:15:1-CHCl₃:MeOH:NH₄OH); ¹H NMR [600 MHz, CDCl₃] δ 3.90 (br s, 1H), 3.28 (d, 1H, J=8.4 Hz), 2.26 (br s, 4H), 1.84 (app t, 1H, J=11.1), 1.55-1.1.38 (m, 4H), 1.36-1.20 (m, 21H), 0.88 (s, 3H), 0.66-0.56 (m, 3H), 0.52-0.46 (m, 1H); ¹³C NMR [100 MHz, CDCl₃] δ 74.3, 69.1, 39.5, 38.5, 37.6, 32.1, 29.88, 29.83, 29.5, 26.1, 22.9, 14.7, 14.3, 12.0 ppm; HRMS [ESI] m/z 314.30516 (calc'd for C₁₉H₄₀NO₂+H: 314.30536); IR (ATR) 3317 (br), 2916, 2849, 1468, 1326, 1072, 1045, 1024, 888, 843, 719, 656 cm⁻¹.

Anti diol (ESPD-0859): R_{f}=0.15 (84:15:1-CHCl₃:MeOH:NH₄OH); ¹H NMR [600 MHz, CDCl₃] δ 3.86-3.82 (m, 1H), 3.18 (d, 1H, J=9.0 Hz), 2.78 (br s, 4H), 1.70-1.62 (m, 2H), 1.53-1.38 (m, 3H), 1.35-1.22 (m, 21H), 0.88 (t, 3H, J=7.2 Hz), 0.67-0.61 (m, 2H), 0.60-0.55 (m, 1H), 0.47-0.44 (m, 1H); ¹³C NMR [100 MHz. CDCl₃] δ 78.6, 72.2, 39.2, 38.5, 38.4, 32.1, 29.87, 29.82, 25.6, 22.9, 14.8, 14.3, 11.6 ppm; HRMS [ESI] m/z 314.30516 (calc'd for C₁₉H₄₀NO₂+H: 314.30536); IR (ATR) 3265 (br), 2917, 2850, 1466, 1342, 1080, 1024, 880, 858, 721, cm⁻¹.

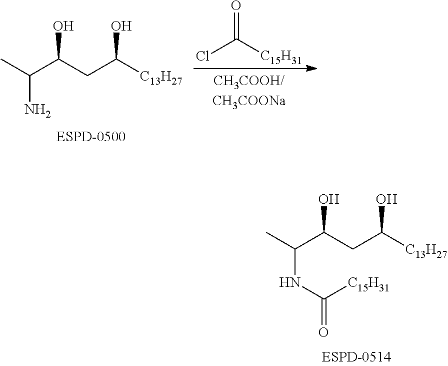

### Preparation of N-((2S,3S,5S)-3,5-dihydroxyoctadecan-2-yl)palmitamide (ESPD-0514)

To a solution of (2S,3S,5S)-2-aminooctadecane-3,5-diol (60 mg, 0.20 mmol) in THF (2.0 mL) and 1M acetic acid (0.1 mL) were added simultaneously palmitoyl chloride (57 mg, 0.21 mmol) and saturated sodium acetate solution, and the resulting reaction mixture was allowed to stir at ambient temperature for 25 min. The mixture was then concentrated under vacuum, and the residue was taken into ether (100 mL). The resulting ether solution was washed with saturated bicarbonate solution (2×10 mL), brine (20 mL), dried over magnesium sulfate, and concentrated under vacuum. Residue was chromatographed on silica gel column with a mixture CHCl₃-MeOH-Acetone, 20:0.5:0.5 to give 71 mg (66.1%) as a white solid. R_{f}=0.52 (H-EA, 1:1); [α]_{D}²⁴=−4.6° (c=0.69, CHCl₃); ¹H NMR [400 MHz, CDCl₃] δ 5.84 (d, J=8.8, 1H), 3.96-3.82 (m, 3H), 2.19 (t, J=7.6 Hz, 2H), 1.65-1.44 (m, 6H), 1.26 (m, 46H), 1.21 (d, J=6.8, 3H), 0.88 (t, J=6.8 Hz, 6H); ¹³C NMR [100 MHz, CDCl₃] δ 173.5, 75.6, 73.3, 49.6, 40.3, 38.7, 37.2, 32.1, 29.91, 29.88, 29.80, 29.58, 29.52, 26.1, 25.5, 22.9, 18.4, 14.3 ppm; HRMS [ESI] m/z 540.53502 (calc'd for C₃₄H₆₉NO₃+H: 540.53502); IR (in mineral oil) 3365, 3304, 1643, 1549, 1350, 1275, 1120, 1079, 860 cm⁻¹.

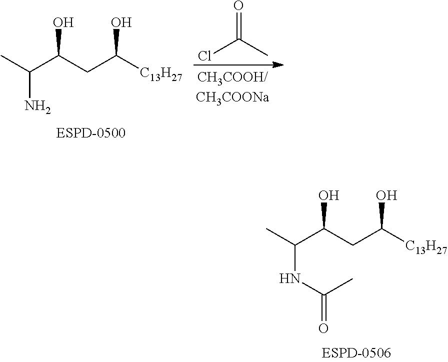

### Preparation of N-((2S,3S,5S)-3,5-dihydroxyoctadecan-2-yl)acetamide (ESPD-0506)

Following the procedure detailed in the synthesis of ESPD-0514 yielded the desired product as a white solid (55 mg, 81%). R_{f}=0.37 (CHCl₃-MeOH-Acetone 10:1:1); [α]_{D}²⁴=−12.4° (c=1.83, CHCl₃) and −22.2° (c=1.83, MeOH); ¹H NMR [400 MHz, CDCl₃] δ 5.94 (d, J=8.8 Hz, 1H), 4.29 (s, 1H), 3.96-3.81 (m, 5H), 2.91 (s, 1H), 2.00 (s, 3H), 1.58-1.42 (br m, 4H), 1.25 (m, 22H), 1.21 (d, J=6.8, 3H), 0.88 (t, J=6.8, 3H); ¹³C NMR [100 MHz, CDCl₃] δ 170.4, 75.5, 73.2, 49.8, 40.2, 38.6, 32.1, 29.86, 29.79, 29.56, 25.5, 23.7, 22.9, 18.3, 14.3 ppm; HRMS [ESI] m/z 344.31598 (calc'd for C₂₀H₄₁NO₃+H: 344.31592); IR (in mineral oil) 3350, 3309, 1646, 1540, 1292, 1120, 1083, 980, 814 cm⁻¹.

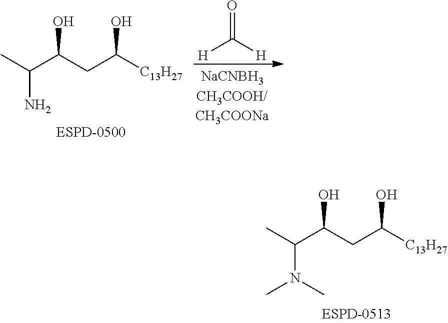

### Preparation of (2S,3S,5S)-2-(dimethylamino)octadecane-3,5-diol (ESPD-0513)

To a solution of (2S,3S,5S)-2-aminooctadecane-3,5-diol (128 mg, 0.42 mmol) in acetate buffer (1.1 g of NaOAc and 0.8 ml of AcOH in 8 mL of water), 1.5 mL of 37% aqueous formaldehyde was added, and the resulting reaction mixture was allowed to stir at ambient temperature for 25 min. Sodium cyanoborohydride (190 mg, 3.0 mmol) and MeOH (2 mL) were added, and stirring was continued for 2 hrs. The reaction mixture was diluted with ether (100 mL), ether solution was washed with saturated sodium bicarbonate (20 ml), brine (20 mL), dried over magnesium sulfate, and concentrated under vacuum. The resulting residue was chromatographed with a mixture CHCl₃-MeOH-25% NH₄OH (100:10:1) to yield 90 mg (64%) of the target compound as an oil. R_{f}=0.52 (CHCl₃-MeOH-25% NH₄OH, 45:10:1) and 0.44 (CHCl₃-MeOH-AcOH-H₂O 45:5:2:0.8); [α]_{D}²⁴=+0.5° (c=1.0, CHCl₃) and −7.1° (c=1.0, MeOH); ¹H NMR [400 MHz, CDCl₃] δ 4.4-4.0 (br m, 2H), 3.88 (m, 1H), 3.45 (m, 1H), 2.41 (m, 1H), 2.24 (s, 6H), 1.67 (m, 0.5H), 1.65 (d, J=14 Hz, 1H), 1.54-1.47 (m, 1H), 1.44-1.38 (m, 2H), 1.34-1.24 (m, 22H), 0.89 (d, J=6.4 Hz, 3H), 0.88 (t, J=6.8 Hz, 3H); ¹³C NMR [100 MHz, CDCl₃] δ 72.5, 72.2, 64.6, 40.4, 40.0, 37.9, 32.1, 29.97, 29.89, 29.57, 25.7, 22.9, 14.3, 7.1 ppm; HRMS [ESI] m/z 330.33666 (calc'd for C₂₀H₄₃NO₂+H: 330.33682); IR (neat) 3364 (br), 2924, 2853, 1733, 1670, 1558, 1540, 1457, 1377, 1298, 1099, 1045, 915, 845 cm⁻¹.

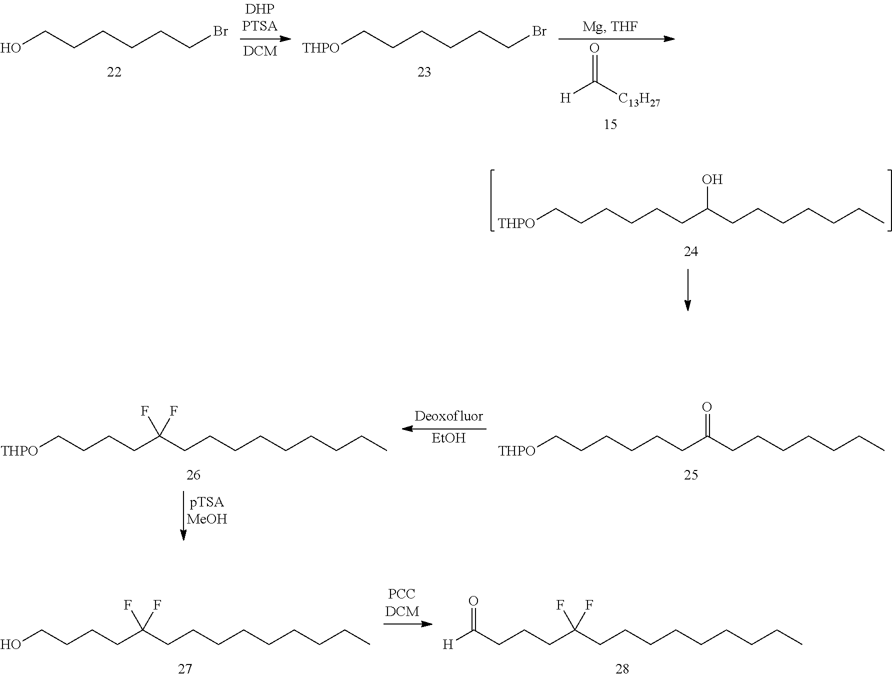

### Preparation of 2-(6-bromohexyloxy)tetrahydro-2H-pyran (23)

6-bromohexan-1-ol (22) (7.25 ml, 55.2 mmol) was brought up in DCM (55 mL) and cooled to 0° C. To this solution was added tosylic acid (0.095 g, 0.55 mmol), then dihydropyran (6.31 ml, 69.0 mmol) was added drop wise. Upon completion of addition, the reaction was stirred for 1.5 hours allowing it to warm to ambient temperature. TLC showed complete conversion to protected alcohol. The mixture was diluted with ether (300 mL), and washed with sat NaHCO₃. The organic layer was dried over MgSO₄ and concentrated in vacuo. The crude product was purified by column chromatography (100% DCM) to give the product as a clear colorless oil (12.05 g, 82%). R_{f}=0.74 (2:1 hexanes to EtOAc); ¹H NMR [400 MHz, CDCl₃] δ 4.55 (dd, 1H, J=4.3 Hz), 3.84 (m, 1H), 3.71 (dt, 1H, J=9.4, 7.0 Hz), 3.47 (m, 1H), 3.38 (t, 2H, J=7.1 Hz), 3.36 (dt, 1H, J=9.4, 7.0 Hz), 1.85 (q, 2H, J=7.2 Hz), 1.80-1.30 (m, 12H); ¹³C NMR [100 MHz, CDCl₃] δ 99.2, 99.0, 67.6, 62.6, 34.1, 32.9, 31.0, 29.8, 28.2, 25.7, 19.9 ppm; HRMS [ESI] m/z 271.00278 (calc'd for C₁₁H₂₁BrO₂+Li: 271.08795); Elem. Anal. C, 50.17; H, 8.00; Br, 29.80 (calc'd for C₁₁H₂₁BrO₂: C, 49.82; H, 7.98; Br, 30.13).

### Preparation of 1-(tetrahydro-2H-pyran-2-yloxy)tetradecan-7-ol (24)

Powdered magnesium (217 mg, 8.9 mmol) was brought up in THF (6 mL) in a flame dried round bottom equipped with a condenser. To this flask was added a portion of 2-(6-bromohexyloxy)tetrahydro-2H-pyran (23) (200 mg, 0.75 mmol), dissolved in THF (6.00 mL) over 2 minutes. The mixture was heated to reflux, and a crystal of iodine was added. The yellow reaction turned colorless after 15 minutes, at which time the remainder of the 2-(6-bromohexyloxy)tetrahydro-2H-pyran (23) was added (1.80 g, 6.0 mmol) via syringe pump. To ensure complete formation of the Grignard, the reaction was stirred at 50° C. overnight. The solution was cooled to room temperature and added dropwise via canula over 20 minutes to a stirring solution of n-octanal (1.03 ml, 6.9 mmol) in THF (6.00 mL) turning the solution bright yellow. The reaction was stirred for 2 hours, over which time the color faded and TLC indicated complete consumption of the starting material. The reaction mixture was diluted with Et₂O (50 mL), extracted with water, and 10% solution of NH₄Cl (aq). The aqueous layer was then back extracted with Et₂O (20 mL). The combined organic layers were dried over MgSO₄ and concentrated in vacuo. The crude product was dried under vacuum overnight to yield a clear slightly yellow oil (1.196 g, 55% yield). The crude product was used directly in the next reaction. R_{f}=0.35 (5:1 hexanes to EtOAc); ¹H NMR [400 MHz, CDCl₃] δ 4.58 (dd, 1H, J=4.3 Hz), 3.87 (m, 1H), 3.73 (dt, 1H, J=9.5, 7.0 Hz), 3.58 (m, 1H), 3.51 (m, 1H), 3.36 (dt, 1H, J=9.4, 6.7 Hz), 1.82 (m, 1H), 1.74 (m, 1H), 1.65-1.20 (m, 27H), 0.87 (t, 3H, J=7.0 Hz); ¹³C NMR [100 MHz, CDCl₃] δ 99.1, 72.2, 67.8, 62.6, 37.7, 37.6, 32.0, 31.0, 29.9, 29.9, 29.7, 29.5, 26.4, 25.9, 25.8, 25.7, 22.9, 19.9, 14.3 ppm.

### Preparation of 1-(tetrahydro-2H-pyran-2-yloxy)tetradecan-7-one (25)

PCC (6.0 g, 45 mmol) and Celite (6 g) were brought up DCM (30 mL). To this mixture was added 1-(tetrahydro-2H-pyran-2-yloxy)tetradecan-7-ol (24) (7.0 g, 22 mmol) dissolved in DCM (30 mL) drop wise via syringe pump. The reaction mixture was stirred at room temperature for 90 minutes, at which time TLC nearly complete conversion to product. This mixture was passed through a silica gel plug (30 g), which was washed with a solution of 5:1 hexanes to EtOAc (200 mL). The crude product obtained was then purified by column chromatography (0-20% EtOAc:Hex) to yield the product as a colorless oil (5.908, 85% yield). R_{f}=0.47 (5:1 Hex: EtOAc); ¹H NMR [400 MHz, CDCl₃] δ 4.56 (dd, 1H, J=4.7 Hz), 3.85 (m, 1H), 3.71 (dt, 1H, J=9.7, 6.7), 3.50 (m, 1H), 3.36 (dt, 1H, J=9.7, 6.4 Hz), 2.38 (t, 2H, J=7.3 Hz), 2.38 (t, 2H, J=7.4 Hz), 1.80 (m, 1H), 1.70 (m, 1H), 1.60-1.45 (m, 10H), 1.40-1.20 (m, 12H), 0.86 (t, 3H, J=7.0 Hz); ¹³C NMR [100 MHz, CDCl₃] δ 211.8, 99.0, 67.7, 62.5, 43.0, 42.9, 31.9, 31.0, 29.8, 29.4, 29.3, 29.3, 26.3, 25.7, 24.1, 24.0, 22.8, 19.9, 14.2 ppm; HRMS [ESI] m/z=313.27384 (calc'd for C₁₉H₃₆O₃+H: 312.26645); IR (ATR) 2927, 2855, 1713, 1458, 1353, 1200, 1135, 1121, 1076, 1031, 987, 904, 869, 814, 723 cm⁻¹; Elem. Anal. C, 72.69: H, 11.64 (calc'd for C₁₉H₃₆O₃: C, 73.03; H, 11.61).

### Preparation of 2-(5,5-difluorotetradecyloxy)tetrahydro-2H-pyran (26)

In an vacuum dried, argon purged plastic bottle (40 mL) capped with a septum and containing a stir bar, 1-(tetrahydro-2H-pyran-2-yloxy)tetradecan-7-one (25) (5.0 g, 16 mmol) was dissolved in DCM (15 mL). To this Deoxofluor (Bis(2-methoxyethyl)aminosulfur trifluoride) (8.9 ml, 48 mmol) diluted with DCM (15 mL), was added drop wise at room temperature, followed by 200 proof ethanol (0.28 ml, 4.8 mmol) to initiate the generation of 0.3 eq. of HF. The reaction was stirred for 90 minutes at which time TLC showed 95% conversion to the desired product. The reaction was quenched by cooling with an ice bath followed by drop wise addition of sat. aq. NaHCO₃ (150 mL) through an addition funnel. The organic layer was separated, then washed again with sat. aq. NaHCO₃ (20 mL), and all aqueous layers were back extracted with DCM. The combined organic layers (˜100 mL) were dried over MgSO₄ and concentrated in vacuo. The crude product was purified by column chromatography (0-20% EtOAc: Hex) to give the product as a clear colorless waxy solid (12.05 g, 82%). Note: elimination product (fluoroalkene) was also observed in approximately 10% yield, and was separated with difficult during chromatography. R_{f}=0.70 (5:1, Hex:EtOAc); ¹H NMR [400 MHz, CDCl₃] δ 4.57 (dd, 1H, J=4.7 Hz), 3.87 (m, 1H), 3.74 (dt, 1H, J=9.7, 6.7 Hz), 3.50 (m, 1H), 3.38 (dt, 1H, J=9.7, 6.4 Hz), 1.90-1.60 (m, 6H), 1.60-1.20 (m, 22H), 0.88 (t, J=7.0 Hz, 3H); ¹³C NMR [100 MHz, CDCl₃[δ 125.6 (t, J=240 Hz), 99.1, 67.7, 62.6, 36.5 (t, J=31.8 Hz), 36.4 (t, J=31.8 Hz), 31.9, 31.0, 29.8, 29.6, 29.5, 29.3, 26.3, 25.7, 22.8, 22.6 (t, J=6.4 Hz), 22.5 (t, J=6.3 Hz), 19.9, 14.3 ppm; ¹⁹F NMR [400 MHz, CDCl₃] δ 98.42 (pentet, J=16.8 Hz); HRMS [ESI] m/z=341.30495 (calc'd for C₁₉H₃₆F₂O₂+Li: 341.28379); IR (ATR) 2929, 2856, 1466, 1382, 1352, 1323, 1260, 1200, 1135, 1120, 1078, 1033, 1022, 962, 904, 869, 841 cm⁻¹; Elem. Anal. C, 68.38; H, 10.86; F. 10.94. (calc'd for C₁₉H₃₆F₂O₂: C, 68.23; H, 10.85; F, 11.36).

### Preparation of 5,5-difluorotetradecan-1-ol (27)

2-(7,7-difluorotetradecyloxy)tetrahydro-2H-pyran (26) (760 mg, 2.3 mmol) was dissolved in MeOH (5 mL) and stirred with tosylic acid (59 mg, 0.34 mmol) overnight at room temperature, at which time the reaction was complete by TLC. Solvent was removed under reduced pressure, and the crude product was purified by flash chromatography (0-20% EtOAc:Hex) to give a waxy white solid (455 mg, 80% yield). R_{f}=0.20 (5:1, Hex:EtOAc); ¹H NMR [400 MHz, CDCl₃] δ 3.65 (t, 2H, J=6.7 Hz), 1.9-1.7 (m, 4H), 1.58 (m, 2H), 1.5-1.2 (m, 17H), 0.89 (t, 3H, J=7.0 Hz); ¹³C NMR [125 MHz, CDCl₃] δ 125.6 (t, J=240 Hz), 63.1, 36.5 (t, J=31.3 Hz), 36.4 (t, J=31.3 Hz), 32.8, 31.9, 29.6, 29.4, 29.3, 25.7, 22.8, 22.6 (t, J=6.4 Hz), 22.5 (t, J=6.3 Hz), 14.3 ppm; ¹⁹F NMR (400 MHz, CDCl₃): δ 98.16 (pentet, J=16.8 Hz) ppm; HRMS [ESI] m/z=257.22628 (calc'd for C₁₄H₂₈F₂O+Li: 257.24741); IR (ATR) 3292, 2951, 2927, 2850, 1459, 1429, 1392, 1395, 1334, 1278, 1229, 1221, 1186, 1142, 1071, 1055, 938, 898, 888, 805, 726, 687 cm⁻¹.

### Preparation of 5,5-difluorotetradecanal (28)

Pyridinium chlorlochromate (1.47 g, 10.8 mmol) and Celite (1.2 g) were taken up into DCM (10.0 ml). To this mixture was added 7,7-difluorotetradecan-1-ol (27) (1.8 g, 7.2 mmol) dissolved in DCM (10 ml) drop wise. The reaction mixture was stirred for 4 hours at which time the TLC showed 90% conversion. The mixture was passed through a plug of silica gel (40 g), which was washed with DCM (200 mL) to yield the final product after evaporation of solvent as a waxy white solid (1.2 g, 67%). The aldehyde was used without further purification. R_{f}=0.65 (5:1, Hex:EtOAc); ¹H NMR [400 MHz, CDCl₃] δ 9.78 (t, 1H, J=1.6 Hz), 2.46 (dt, 2H, J=7.6, 2.0 Hz), 1.9-1.7 (m, 4H), 1.7-1.6 (m, 2H), 1.5-1.2 (m, 14H), 0.89 (t, 3H, J=6.8 Hz); ¹³C NMR [125 MHz, CDCl₃] δ 202.7, 125.5 (t, J=240 Hz), 43.9, 36.6 (t, J=25.4 Hz), 36.2 (t, J=25.4 Hz), 31.9, 29.6, 29.3, 29.1, 22.8, 22.5, 22.3, 22.0, 14.3 ppm; ¹⁹F NMR [400 MHz, CDCl₃] δ −98.40 (pentet, J=17.6 Hz). IR (ATR) 2928, 2857, 1726, 1389, 1142, 956, 733 cm⁻¹.

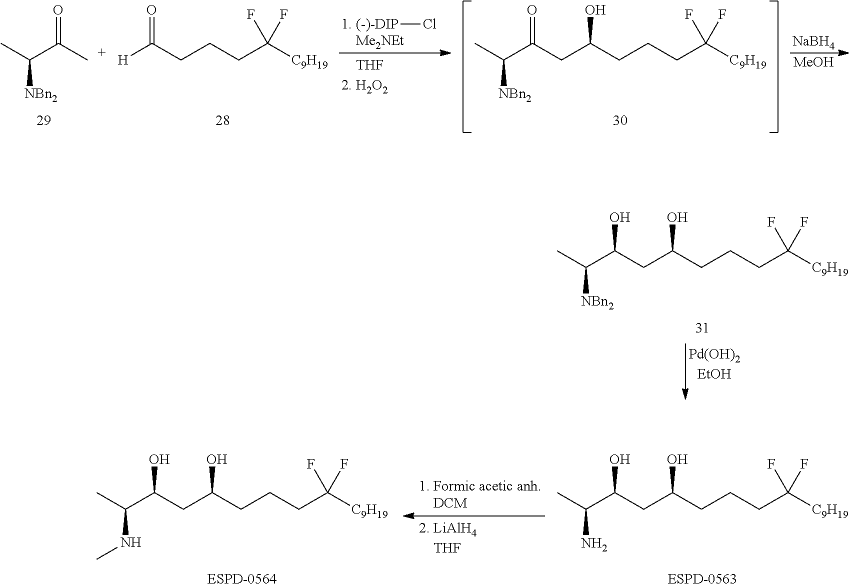

### Preparation of (2S,3S,5S)-2-(dibenzylamino)-9,9-difluorooctadecane-3,5-diol (31)

To a stirred solution of (−)-DIP-Cl (chlorobis((2S,3R)-3,6,6-trimethylbicyclo [3.1.1]heptan-2-yl)borane) (4.4 ml, 6.4 mmol) in THF (20 mL) was added newly purchased N,N-dimethylethanamine (2.0 ml, 19.2 mmol) drop wise at room temperature. The reaction mixture was cooled to −30° C., and a solution of (S)-3-(dibenzylamino)butan-2-one (29) (1.28 g, 4.8 mmol) in THF (10 ml) was added drop wise over a 15 minute period. The resulting mixture was stirred for 1.5 hrs at −30° C., then cooled to −78° C. A solution of freshly prepared 7,7-difluorotetradecanal (28) (1.21 g, 4.9 mmol) in THF (15 mL) was added dropwise over the next 2 hours and maintained at −78° C. overnight. The reaction mixture was quenched by warming to −20° C. and adding phosphate buffer (10 mL, pH=7) over 3 minutes. Stirring was continued over the next hour while the reaction was allowed to warm to room temperature. The mixture was again cooled to −20° C., and hydrogen peroxide (30%) (1.3 mL, 13 mmol) was added drop wise to the stirring mixture. After stirring for an additional hour at −20° C., the reaction mixture was diluted into Et₂O (500 mL), washed with brine (50 mL), dried over MgSO₄, and concentrated under in vacuo to give the crude intermediate (30) (5.53 g), which was immediately taken on to the next step. R_{f}=0.45 (5:1, hexanes:EtOAc); LCMS shows m/z=488.4 (M+H).

Reduction of the 1,3-ketoalcohol was achieved by dissolving the crude (2S,5S)-2-(dibenzylamino)-11,11-difluoro-5-hydroxyoctadecan-3-one (30) (5.53 g) in MeOH (100 mL) and cooling to −20° C. To this was added sodium borohydride (200 mg, 9.08 mmol) in small portions until no more bubbling was observed. To quench any excess hydride, acetic acid (0.5 mL) was added dropwise to the stirring solution. The crude mixture was then concentrated in vacuo, diluted with ether (600 mL) and neutralized with a few drops of saturated sodium bicarbonate until no bubbling was observed. Water (250 mL) was added to the mixture and the organic layer was separated, washed with brine (200 mL), dried over MgSO₄, and concentrated in vacuo to give the crude product as a yellow oil. This residue was subject to sublimation at 0.2 mm Hg at 50° C. and stirring overnight to remove pinene-based byproducts (1.6 g, white crystalline solid). The resulting oil was purified by column chromatography (0-12% EtOAc:Hex w/ 2% Et₃N) resulting in a slightly opaque colorless oil (800 mg, 34% yield). R_{f}=0.35 (5:1, Hex:EtOAc), 4% H₂SO₄ stain); [α]_{D}²⁴+27.8° (c=1.09 in CHCl₃), +7.0° (c=1.09 in MeOH); ¹H NMR [400 MHz, CDCl₃] δ 7.32 (m, 4H), 7.26 (m, 6H), 4.77 (bs, 1H), 4.07 (bs, 1H), 3.84 (m, 1H), 3.81 (d, 2H, J=13.2 Hz), 3.65 (dt, 1H, J=9.2, 1.6 Hz), 3.30 (d, 2H, J=13.2 Hz), 2.54 (m, 1H), 2.74 (ap, 1H, J=6.4 Hz), 1.77 (m, 4H), 1.56 (dt, 1H, J=14.4, 2.4, Hz), 1.5-1.2 (m, 18H), 1.01 (d, 3H, J=6.8 Hz), 0.88 (t, 3H, J=6.8 Hz); ¹³C NMR [125 MHz, CDCl₃] δ 138.8 (2C), 129.2 (4C), 128.8 (4C), 127.6 (2C), 125.7 (t, J=240 Hz), 72.2, 72.0, 59.0, 53.4, 40.3, 37.7, 36.5 (t, J=11.4 Hz), 36.4 (t, J=11.4 Hz), 31.9, 29.7, 29.6, 29.3 (2C), 25.4, 22.8, 22.6 (m, 2C), 14.3, 8.2 ppm; ¹⁹F NMR [400 MHz, CDCl₃] δ −97.50 (p, J=17.6 Hz); HRMS [ESI] m/z 518.38001 (calc'd for C₃₂H₄₉NO₂+H: 518.38041); IR (ATR) 3510, 3370, 3064, 3023, 2928, 2855, 1495, 1454, 1379, 1302, 1142, 1098, 1058, 1028, 972, 845, 748, 698, 500 cm⁻¹; Elem. Anal. C, 74.25; H, 9.40; F, 7.08; N, 2.80 (calc'd for C₃₂H₄₉NO₂: C, 74.24; H, 9.54; F, 7.34; N, 2.71).

### Preparation of (2S,3S,5S)-2-amino-9,9-difluorooctadecane-3,5-diol (ESPD-0563)

(2S,3S,5S)-2-(dibenzylamino)-11,11-difluorooctadecane-3,5-diol (31) (475 mg, 0.92 mmol) and palladium (II) hydroxide (213 mg, 0.30 mmol) were brought up in 190 proof ethanol (30 mL). The heterogeneous solution was purged of air under water aspirated vacuum (˜10 mm Hg) 4 times and filled with argon each time. The evacuated flask was filled with hydrogen, evacuated, then again filled with hydrogen gas (1 atm) and left to react with intense stirring for 1 hour at which point TLC showed complete conversion to the product. The heterogenous mixture was filtered through Celite and the cake was washed with ethanol (30 mL). The solvent was evaporated under reduced pressure to give a white solid as a crude product, which was purified by column chromatography (100% DCM to 100% of 84:15:1 DCM:MeOH:NH₄OH (30% aq.)) to give the final product as a white powdery solid (271.8 mg, 88% yield). R_{f}=0.31 (84:15:1, DCM:MeOH:NH₄OH (30% aq)); mp=69-70° C.; IR (ATR): 3391, 3326, 3277, 2957, 2929, 2853, 2807, 1589, 1462, 1328, 1188, 1167, 1018, 974, 897, 806 cm⁻¹. [α]_{D}²⁴=+2.6° (c=1.03 in CHCl₃),−8.5° (c=1.03 in MeOH); ¹H NMR [400 MHz, CDCl₃] δ 3.85 (m, 1H), 3.45 (m, 1H), 2.85 (bs, 4H), 2.74 (m, 1H), 1.78 (m, 4H), 1.62 (dt, 1H, J=14.4, 2.4 Hz), 1.5-1.2 (m, 19H), 1.10 (d, 3H, J=6.4 Hz), 0.88 (t, 3H, J=6.8 Hz); ¹³C NMR [125 MHz, CDCl₃] δ 125.6 (t, J=240 Hz), 76.7, 71.9, 51.8, 40.4, 38.0, 36.5 (t, J=31.6 Hz), 36.5 (t, J=31.4 Hz), 31.9, 29.9 (m), 29.6, 29.5, 29.3, 25.4, 22.8, 22.5 (m), 21.0, 14.3 ppm; ¹⁹F NMR [400 MHz, CDCl₃] δ −97.80 (p, J=17.6 Hz); HRMS [ESI] m/z=338.28629 (calc'd for C₁₈H₃₇FNO₂+H: 338.28651); Elem. Anal. C, 64.02; H, 10.87; F, 11.09; N, 4.06 (calcd. for C₁₈H₃₇FNO₂: C, 64.06; H, 11.05; F, 11.26; N. 4.15).

### Preparation of (2S,3S,5S)-9,9-difluoro-2-(methylamino)octadecane-3,5-diol (ESPD-0564)

(2S,3S,5S)-2-amino-11,11-difluorooctadecane-3,5-diol (ESPD-0563) (76 mg, 0.22 mmol) was dissolved in DCM (5 mL) and cooled to 0° C. Formic acetic anhydride (40 mg, 0.45 mmol) was added dropwise and the resulting reaction mixture was stirred for 1 hour while warming to room temperature. LCMS and TLC showed conversion to the formamide (R_{f}=0.60 (9:1, DCM:MeOH). The solvent from the reaction was removed under reduce pressure to yield the crude product, which was isolated by column chromatography (0-10% MeOH:DCM) (60 mg, 73% yield). ¹H NMR [400 MHz, CDCl₃] δ 8.17 (s, 1H), 6.24 (d, 1H, J=8.8 Hz), 4.03 (m, 1H), 3.84 (m, 2H), 1.78 (m, 3H), 1.6-1.2 (m, 24H), 0.88 (t, 3H, J=7.0 Hz); ¹³C NMR [125 MHz, CDCl₃] δ 161.4, 125.6 (t, J=240 Hz), 74.9, 72.8, 48.4, 40.0, 38.3, 36.5 (t, J=25.4 Hz), 36.3 (t, J=25.4 Hz), 31.9, 29.9, 29.5, 29.3, 25.2, 22.8, 22.5, 18.3, 14.3 ppm; HRMS [ESI] m/z=366.28154 (calc'd for C₁₉H₃₉F₂NO₂+H: 366.28143).

The formamide (60 mg, 0.164 mmol) was dissolved in THF (5 mL) and cooled to −20° C. and lithium aluminium hydride (1.0 M in THF, 0.49 ml, 0.49 mmol) was added drop wise. The reaction was then slowly warmed up to room temperature. The reaction was quenched with the drop wise addition of 1M NaOH (0.2 mL). The solvent was then slowly removed under reduced pressure, and the resulting solids were then brought up in water (10 mL) and extracted with DCM (4×15 mL). The combined organic layers were dried over MgSO₄ and evaporated to give a crude oil which solidified upon standing to give a waxy solid, which was purified by column chromatography (100% DCM to 100% of a 84:15:1 mix of DCM:MeOH:NH₄OH (30% aq.)). The desired compound went from a clear colorless oil to a waxy solid after being subject to high vac. with overnight. R_{f}=0.05 (84:15:1, DCM:MeOH:NH₄OH (30% aq)); [α]_{D}²⁴=+4.8° (c=0.44 in CHCl₃); ¹H NMR [400 MHz, CDCl₃] δ 3.86 (m, 1H), 3.41 (ddd, 1H, J=12.4, 8.0, 2.8 Hz), 2.43 (s, 3H), 2.33 (m, 1H), 1.9-1.7 (m, 4H), 1.70 (dt, 1H, J=14.4, 2.4 Hz), 1.5-1.2 (m, 22H), 1.07 (d, 3H, J=6.4, Hz), 0.89 (t, 3H, J=7.0 Hz); ¹³C NMR [125 MHz, CDCl₃] δ 125.7 (t, J=240.1), 75.5, 71.6, 60.2, 40.7, 37.9, 36.5 (t, J=25.4 Hz), 36.3 (t, J=25.4 Hz), 33.7, 31.9, 29.9, 29.7, 29.6, 29.2, 25.5, 22.8, 22.6 (m), 16.0, 14.3 ppm; ¹⁹F NMR [400 MHz, CDCl₃] δ −98.05 (p, J=17.6 Hz); HRMS [ESI[m/z=352.30226 (calc'd for C₁₉H₃₉F₂NO₂+H: 352.30216); IR (ATR): 3322, 2922, 2853, 1461, 1378, 1139, 972, 890, 724 cm⁻¹.

### Preparation of 2-amino-6,6-difluorooctadecane-3,5-diols

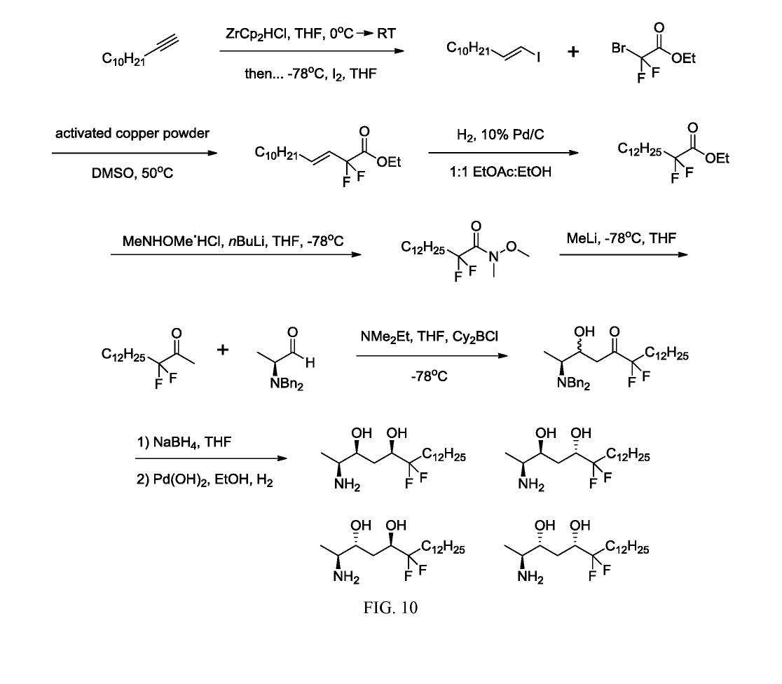

An illustration for the preparation of 2-amino-6,6-difluorooctadecane-3,5-diols is provided in FIG. 10.

### (E)-1-iodododec-1-ene

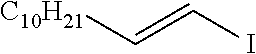

Schwartz reagent (ZrCp₂HCl, 6.92 g, 26.8 mmol, 1.6 eq) was added to a flame-dried flask with stir bar. The solid was diluted with 78 mL THF, cooled to 0° C., and stirred under Ar. Dodec-1-yne (3.59 ml, 16.77 mmol, 1.0 eq) was added dropwise at 0° C., and the resulting mixture was allowed to warm to room temperature and to stir for 3.5 hrs (solution turned black on this scale). The reaction mixture was cooled to −78° C., and a solution of diiodine (5.11 g, 20.13 mmol, 1.2 eq) in 34 mL THF was added via syringe pump over 50 min. The mixture was stirred for another 2 hrs at −78° C. At this point, the reaction mixture was warmed to 0° C. and poured over 78 mL 0.1N HCl. The aqueous layer was extracted 3 times with 50 mL diethyl ether. The combined organic layers were washed with 50 mL saturated NaHCO₃, 50 mL saturated Na₂S₂O₃, 50 mL brine, dried over MgSO₄, filtered, and evaporated under reduced pressure. Purification was carried out via column chromatography using hexanes to yield a clear oil (4.59 g, 15.59 mmol, 93% yield). R_{f}=0.90 (hexanes). IR (νₘₐₓ, cm⁻¹) 2922, 2852, 1461, 1376, 1197, 943, 721, 660. ¹H NMR (400 MHz, CDCl₃) δ 6.52 (dt, J=7.6 Hz, J=14.4 Hz, 1H), 5.97 (dt, J=1.2 Hz, J=14.4 Hz, 1H), 2.05 (dq, J=1.2 Hz, J=7.6 Hz, 2H), 1.39 (p, J=7.2 Hz, 2H), 1.26 (m, 14H), 0.89 (t, J=6.8 Hz, 3H). ¹³C NMR (100 MHz, CDCl₃) δ 146.8, 74.5, 36.3, 32.1, 29.8, 29.7, 29.6, 29.5, 29.1, 28.6, 22.9, 14.3. HRMS (ESI) m/z=295.09178 (Theo. for C₁₂H₂₃I+H: 295.09173).

### (E)-ethyl 2,2-difluorotetradec-3-enoate

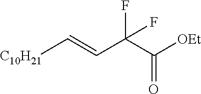

Activated copper powder (17.00 g, 268 mmol, 3.1 eq) was added to a 1 L 3-neck flame-dried flask with stir bar. 292 mL DMSO (Dry-Solv) was added, and the resulting solution was stirred under Ar. A solution of (E)-1-iodododec-1-ene (25.28 g, 86.0 mmol, 1.0 eq) in 138 mL DMSO (Dry-Solv) was added via addition funnel. Ethyl 2-bromo-2,2-difluoroacetate (11.02 mL, 86.0 mmol, 1.0 eq) was added via syringe to vigorously stirring solution. The reaction flask was equipped with a reflux condenser, warmed to 55° C., and stirred overnight under Ar. In the morning, the mixture was poured over a cold 1:1 solution of saturated ammonium chloride solution to water. The aqueous layer was extracted with diethyl ether 4 times. The combined organic layers were washed twice with saturated ammonium chloride solution and twice with brine, dried over MgSO₄, filtered, and evaporated under reduced pressure. The resulting crude oil was purified via column chromatography using 1:40 EtOAc to hexanes to yield a clear oil (17.31 g, 59.6 mmol, 69% yield). R_{f}=0.40 (1:40 EA/H), R_{f}=0.45 (1:20 EA/H). IR (νₘₐₓ, cm⁻¹) 2925, 2855, 1767, 1674, 1465, 1372, 1292, 1227, 1079, 969, 854, 780, 721. ¹H NMR (400 MHz, CDCl₃) δ 6.27 (m, 1H), 5.67 (m, 1H), 4.33 (q, J=7.2 Hz, 2H), 2.14 (m, 2H), 1.42 (p, J=7.2 Hz, 2H), 1.36 (t, J=7.2 Hz, 3H), 1.27 (m, 14H), 0.89 (t, J=6.8 Hz, 3H). ¹³C NMR (100 MHz, CDCl₃) δ 164.4 (t, J=34.6 Hz), 140.2 (t, J=8.9 Hz), 121.1 (t, J=25.0 Hz), 112.6 (t, J=246 Hz), 63.0, 32.1 (2C), 29.8, 29.7, 29.6, 29.5, 29.2, 28.3, 22.9, 14.3, 14.1. ¹⁹F NMR (376 MHz, CDCl₃) 6-103.2 (dd, J=3.0 Hz, J=5.6 Hz, 1F), −103.2 (dd, J=3.0 Hz, J=5.6 Hz, 1F). HRMS (ESI) m/z=291.21313 (Theo. for C₁₆H₂₈O₂F₂+H: 291.21301). Anal. Calcd. for C₁₆H₂₈O₂F₂: C, 66.18; H, 9.72; F, 13.08. Found: C, 65.98; H, 9.50; F, 12.84.

### Ethyl 2,2-difluorotetradecanoate

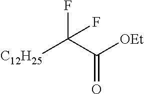

A solution of (E)-ethyl 2,2-difluorotetradec-3-enoate (3.056 g, 10.5 mmol, 1.0 eq) in 132 mL EtOAc was added to a 1 L flask with stir bar. The solution was further diluted with 132 mL EtOH, and the mixture was stirred vigorously under Ar. 10% palladium on carbon (1.120 g, 1.052 mmol, 0.10 eq) was added, and the resulting mixture was stirred vigorously under Ar at room temperature. The flask was evacuated, and then filled with Ar. These 2 steps were repeated twice more. Finally, one last evacuation was carried out, and the flask was filled with H₂ via balloon. The resulting mixture was stirred vigorously at room temperature under H₂ for 24 hours. The reaction was monitored by LCMS. Upon consumption of starting material, the reaction mixture was filtered over celite, and solvent was evaporated under reduced pressure. Purified via column chromatography using 1:40 EtOAc to hexanes to yield a slightly yellow oil (2.89 g, 9.88 mmol, 94% yield). R_{f}=0.59 (1:20 EA/H), R_{f}=0.75 (1:10 EA/H). IR (νₘₐₓ, cm⁻¹) 2924, 2854, 1764, 1465, 1374, 1336, 1304, 1189, 1130, 1095, 1034, 849, 778, 724, 642. ¹H NMR (400 MHz, CDCl₃) δ 4.33 (q, J=6.8 Hz, 2H), 2.06 (m, 2H), 1.46 (app p, J=7.6 Hz, 2H), 1.36 (t, J=7.2 Hz, 3H), 1.27 (m, 18H), 0.89 (t, J=6.8 Hz, 3H). ¹³C NMR (100 MHz, CDCl₃) δ 164.6 (t, J=33.1 Hz), 116.6 (t, J=248.6 Hz), 62.8, 34.7 (t, J=23.1 Hz), 32.1, 29.8, 29.8, 29.8, 29.6, 29.6, 29.4, 29.3, 22.9, 21.6 (t, J=4.1 Hz), 14.3, 14.1. ¹⁹F NMR (376 MHz, CDCl₃) 6-106.5 (t, J=16.9 Hz, 2F). HRMS (ESI) m/z=293.22788 (Theo. for C₁₆H₃₀O₃+H: 293.22866).

### 2,2-difluoro-N-methoxy-N-methyltetradecanamide

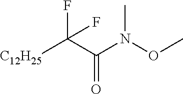

N,O-dimethylhydroxylammonium chloride (7.24 g, 74.2 mmol, 3.0 eq) was added to a flame-dried 1 L flask with stir bar. The solid was diluted with 132 mL THF, cooled to −78° C., and stirred under Ar. n-Butyllithium (59.3 mL, 148 mmol, 6.0 eq) was added dropwise via syringe pump over 90 min at −78° C., and the resulting solution was allowed to stir at −78° C. for 10 min. The dry ice-acetone bath was removed for 10 min, and the reaction mixture was subsequently recooled to −78° C. A solution of ethyl 2,2-difluorotetradecanoate (7.23 g, 24.7 mmol, 1.0 eq) in 115 mL THF was added dropwise via cannula at −78° C., and the resulting mixture was stirred under Ar at −78° C. 15 min after the addition was complete, TLC indicated consumption of starting material. The reaction was quenched with saturated ammonium chloride at −78° C., and the resulting mixture was allowed to warm above 0° C. A small volume of water was added to dissolve salts. The aqueous layer was extracted 3 times with EtOAc. The combined organic layers were washed with brine, dried over MgSO₄, filtered, and evaporated under reduced pressure. The product was recrystallized from pentane to yield a white solid (6.649 g, 21.63 mmol, 87%). R_{f}=0.35 (1:10 EA/H), R_{f}=0.50 (1:4 EA/H). MP 33-35° C. IR (νₘₐₓ, cm⁻¹) 2917, 2849, 1678, 1461, 1184, 1161, 1016, 968, 876, 725, 653. ¹H NMR (400 MHz, CDCl₃) δ 3.75 (s, 3H), 3.27 (br s, 3H), 2.12 (m, 2H), 1.49 (app p, J=7.6 Hz, 2H), 1.26 (m, 18H), 0.89 (t, J=6.8 Hz, 3H). ¹³C NMR (100 MHz, CDCl₃) δ 164.2 (t, J=23.1 Hz), 118.2 (t, J=248.2 Hz), 61.9, 34.5 (t, J=23.1 Hz), 32.9, 32.0, 29.7, 29.7, 29.7, 29.5, 29.4, 29.4, 29.3, 22.7, 21.5, 14.1. ¹⁹F (376 MHz, CDCl₃) 6-104.1 (app s, 2F). HRMS (ESI) m/z=308.23974 (Theo. for C₁₆H₃ᵢF₂NO₂+H: 308.23956). Anal. Calcd. for C₁₆H₃₁F₂NO₂: C, 62.51; H, 10.16; F, 12.36; N, 4.56. Found: C, 62.58; H, 10.07; F, 12.21; N, 4.56.

### 3,3-difluoropentadecan-2-one

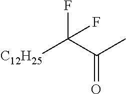

2,2-difluoro-N-methoxy-N-methyltetradecanamide (2.00 g, 6.51 mmol, 1.0 eq) was added to a flame-dried 250 mL 3-neck flask equipped with internal low temperature thermometer and stir bar. The solid was diluted with 65 mL THF, cooled to −78° C., and stirred under Ar. Methyllithium (1.6 M, 5.29 mL, 8.46 mmol, 1.3 eq) was added dropwise via syringe pump at a rate of 0.136 mL/min at −78° C. under Ar. The temperature remained below −63° C. during addition. After addition was complete, TLC indicated consumption of starting material. The reaction was quenched dropwise with saturated ammonium chloride solution at −78° C., and the resulting mixture was allowed to warm above 0° C. THF was evaporated under reduced pressure. The remaining aqueous layer was diluted with a small volume of water to dissolve salts, and extracted 3 times with EtOAc. The combined organic layers were washed twice with brine, dried over MgSO₄, filtered, and evaporated under reduced pressure. Crude oil was purified via column chromatography using 1:50 EtOAc to hexanes to yield a clear oil (1.339 g, 5.10 mmol, 78%). R_{f}=0.82 (1:10 EA/H), R_{f}=0.60 (1:30 EA/H), R_{f}=0.49 (1:40 EA/H). IR (νₘₐₓ, cm¹) 2924, 2854, 1747, 1465, 1361, 1206, 1137, 1034, 984. ¹H NMR (400 mHz, CDCl₃) δ 2.33 (t, J=1.6 Hz, 3H), 1.97 (m, 2H), 1.44 (m, 2H), 1.26 (m, 18H), 0.89 (t, J=6.8 Hz, 3H). ¹³C NMR (100 MHz, CDCl₃) δ 199.2 (t, J=33.1 Hz), 118.3 (t, J=249.7 Hz), 32.5 (t, J=22.7 Hz), 32.1, 29.8, 29.8, 29.8, 29.6, 29.6, 29.5, 29.5, 24.1, 22.9, 21.4 (t, J=4.1 Hz), 14.2. ¹⁹F (376 MHz, CDCl₃) 6-107.6 (dt, J=17.5 Hz, 1.5 Hz, 2F). Anal. Calcd. for C₁₅H₂₈F₂O: C, 68.66; H, 10.76; F, 14.48. Found: C, 68.78; H, 10.74; F, 14.62.

### (2S,3S)-2-(dibenzylamino)-6,6-difluoro-3-hydroxyoctadecan-5-one

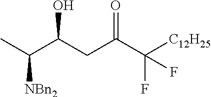

N,N-dimethylethanamine (1.22 mL, 11.3 mmol, 4.0 eq) added to flame-dried flask with stir bar. Diluted with 8.0 mL THF, cooled to −78° C., and stirred under Ar. Chlorodicyclohexylborane (1.0 M 3.81 mL, 3.81 mmol, 1.4 eq) was added dropwise, and the mixture was stirred under the same conditions for 30 min. A solution of 3,3-difluoropentadecan-2-one (0.74 g, 2.82 mmol, 1.0 eq) in 12.0 mL THF added slowly, and the resulting mixture was stirred for 30 min under same conditions. The cold bath was removed for 15 min, and then replaced for 10 min. A solution of (S)-2-(dibenzylamino)propanal (0.786 g, 3.10 mmol, 1.1 eq) in 8.0 mL THF was added slowly via syringe pump, and the resulting mixture was stirred at −78° C. under Ar for 2 hours, when the aminoaldehyde positive ion ceased to decrease by LCMS. The reaction was quenched with phosphate buffer (pH=7), and the mixture was allowed to warm to room temperature. The solution was cooled back to −78° C., and a solution of hydrogen peroxide in H₂O (30%, 0.778 mL, 7.61 mmol, 2.7 eq) was added dropwise. The mixture was allowed to warm to room temperature once again, and then diluted with diethyl ether. The organic layer was washed with brine, dried over MgSO₄, filtered, and evaporated under reduced pressure. Chiral HPLC verified that no racemization occurred during the reaction, and LCMS showed a diastereomeric ratio of 61:39 slightly favoring the syn aminoalcohol (2S,3S). Diastereomers were separated by column chromatography (0.712 g, 1.38 mmol, 49%). R_{f}=0.25 (1:15 EA/H), R_{f}=0.35 (1:10 EA/H). [α]_{D}²⁴=+4.3° (c=1.00 in EtOAc). IR (νₘₐₓ, cm⁻¹) 3446, 2924, 2853, 1737, 1454, 1377, 1208, 1149, 1095, 1072, 1048, 1027, 746, 698. ¹H NMR (400 MHz, CDCl₃) δ 7.22-7.39 (m, 10H), 4.07 (app t, J=8.6 Hz, 1H), 3.74 (d, J=14.0 Hz, 2H), 3.44 (app d, J=18.8 Hz, 1H), 3.41 (d, J=13.6 Hz, 2H), 2.67 (dq, J=6.7 Hz, 8.6 Hz, 1H), 2.52 (br s, 1H), 2.47 (dd, J=9.8 Hz, 19.0 Hz, 1H), 1.93 (m, 2H), 1.43 (app p, J=7.6 Hz, 2H), 1.27 (m, 18H), 1.19 (d, J=6.4 Hz, 3H), 0.892 (t, J=6.8 Hz, 3H). ¹³C NMR (100 MHz, CDCl₃) δ 202.7 (t, J=31.7 Hz), 139.7 (2C), 129.0 (4C), 128.5 (4C), 127.2 (2C), 118.3 (t, J=250.4 Hz), 69.0, 66.0, 56.8, 54.6, 42.1, 32.7 (t, J=22.7 Hz), 32.1, 29.8 (2C), 29.6, 29.5, 29.4 (2C), 22.9, 21.3, 15.4, 14.3, 8.4. ¹⁹F NMR (376 MHz, CDCl₃) 6-107.8 (dt, J=17.7 Hz, 271.8 Hz, 1F), −108.6 (dt, J=17.7 Hz, 271.8 Hz, 1F). HRMS (APCI) m/z=516.36529 (Theo. for C₃₂H₄₇F₂NO₂+H: 516.36476). Anal. Calcd. for C₃₂H₄₇F₂NO₂: C, 74.53; H, 9.19; N, 2.72; F, 7.37. Found: C, 74.65; H, 9.08; N, 2.88; F, 7.31.

### (2S,3R)-2-(dibenzylamino)-6,6-difluoro-3-hydroxyoctadecan-5-one

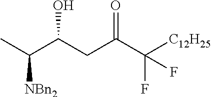

(0.455 g, 0.883 mmol, 31%) R_{f}=0.38 (1:15 EA/H), R_{f}=0.49 (1:10 EA/H). [α]_{D}²⁴=+7.7° (c=1.00 in EtOAc). IR (νₘₐₓ, cm⁻¹) 3407, 2923, 2853, 1744, 1454, 1379, 1208, 1144, 1055, 1027, 1002, 944, 747, 731, 698, 627. ¹H NMR (400 MHz, CDCl₃) δ 7.25-7.35 (m, 10H), 4.35 (br s, 1H), 4.06 (ddd, J=4.7 Hz, 6.7 Hz, 9.4 Hz, 1H), 3.87 (d, J=13.6 Hz, 2H), 3.33 (d, J=13.2 Hz, 2H), 2.67 (m, 1H), 2.65 (m, 1H), 2.64 (m, 1H), 1.94 (m, 2H), 1.44 (app p, J=7.7 Hz, 2H), 1.27 (m, 18H), 1.05 (d, J=6.8 Hz, 3H), 0.898 (t, J=7.0 Hz, 3H). ¹³C NMR (100 MHz, CDCl₃) δ 199.9 (t, J=31.7 Hz), 138.7 (2C), 129.2 (4C), 128.7 (4C), 127.5 (2C), 118.5 (t, J=250.0 Hz), 66.9, 58.0, 53.4, 41.4, 32.5 (t, J=22.3 Hz), 32.1, 30.0 (3C), 29.7, 29.6, 29.5, 29.4 (2C), 22.9, 21.2, 14.3, 8.14. ¹⁹F NMR (376 MHz, CDCl₃) 6-107.9 (dt, J=18.0 Hz, 271.8 Hz, 1F), −108.7 (dt, J=17.9 Hz, 271.8 Hz, 1F). HRMS (APCI) m/z=516.36540 (Theo. for C₃₂H₄₇F₂NO₂+H: 516.36476). Anal. Calcd. for C₃₂H₄₇F₂NO₂: C, 74.53; H, 9.19; N, 2.72; F, 7.37. Found: C, 74.79; H, 9.19; N, 2.90; F, 7.21.

### (2S,3S,5R)-2-(dibenzylamino)-6,6-difluorooctadecane-3,5-diol

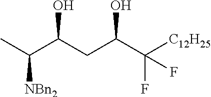

Sodium tetrahydroborate (1.5 eq) was added to a flame-dried flask with stir bar, diluted with THF, and stirred under Ar at room temperature. A solution of 2S-(dibenzylamino)-6,6-difluoro-3-hydroxyoctadecan-5-one (1.0 eq) in THF was added via syringe pump over 30 min. Once starting material disappeared on TLC, the reaction mixture was cooled to 0° C. and quenched dropwise with H₂O. 2N HCl was added dropwise until neutral pH was achieved. The aqueous layer was extracted with EtOAc. Combined organic layers were washed with brine, dried over MgSO₄, filtered, and evaporated under reduced pressure. LCMS showed a 1:1 mixture of diastereomers, which proved to be separable by column chromatography (0.085 g, 0.164 mmol, 28%). R_{f}=0.41 (1:5 EA/H), R_{f}=0.18 (1:10 EA/H). [α]_{D}²⁴=+1.1° (c=1.00 in EtOAc). IR (νₘₐₓ, cm⁻¹) 3405, 2923, 2853, 1454, 1147, 1010, 1073, 1027, 1001, 745, 698. ¹H NMR (400 MHz, CDCl₃) δ 7.23-7.35 (m, 10H), 3.87 (m, 1H), 3.79 (d, J=14.0 Hz, 2H), 3.76 (m, 1H), 3.44 (d, J=13.6 Hz, 2H), 2.79 (p, J=6.9 Hz, 1H), 2.25 (app d, J=14.4 Hz, 1H), 1.77-1.97 (m, 2H), 1.51 (m, 2H), 1.4 (m, 1H), 1.28 (m, 18H), 1.16 (d, J=6.8 Hz, 3H), 0.892 (t, J=6.8 Hz, 3H). ¹³C NMR (100 MHz, CDCl₃) δ 139.7 (2C), 129.1 (4C), 128.6 (4C), 127.4 (2C), 123.8 (t, J=244.1 Hz), 74.5, 74.3 (t, J=28.3 Hz), 57.8, 55.2 (2C), 32.8, 32.4 (t, J=23.8 Hz), 32.1, 29.8 (3C), 29.7 (2C), 29.6, 29.5, 22.9, 21.5, 14.3, 8.7. ¹⁹F NMR (376 MHz, CDCl₃) 6-111.1 (m, 1F), −114.1 (m, 1F). HRMS (APCI) m/z=518.38075 (Theo. for C₃₂H₄₉F₂NO₂+H: 518.38041).

### (2S,3S,5S)-2-(dibenzylamino)-6,6-difluorooctadecane-3,5-diol

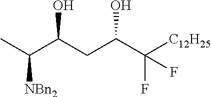

(0.133 g, 0.257 mmol, 44%) R_{f}=0.34 (1:5 EA/H), R_{f}=0.13 (1:10 EA/H). [α]_{D}²⁴=+14.4° (c=1.00 in EtOAc). IR (νₘₐₓ, cm⁻¹) 3360, 2920, 2851, 1453, 1379, 1247, 1205, 1162, 1073, 1028, 1002, 976, 933, 905, 731, 697, 663. ¹H NMR (400 MHz, CDCl₃) δ 7.23-7.35 (m, 10H), 3.93 (td, J=2.1 Hz, 8.0 Hz, 1H), 3.83 (m, 1H), 3.76 (d, J=13.6 Hz, 2H), 3.42 (d, J=14.0 Hz, 2H), 2.78 (app p, J=6.6 Hz, 1H), 2.59 (br s, 1H), 2.31 (br s, 1H), 1.99 (ddd, J=2.7 Hz, 10.0 Hz, 14.4 Hz, 1H), 1.76-1.92 (m, 2H), 1.70 (ddd, J=2.4 Hz, 8.8 Hz, 14.4 Hz, 1H), 1.50 (app p, J=7.5 Hz, 2H), 1.28 (m, 18H), 1.19 (d, J=6.8 Hz, 3H), 0.891 (t, J=6.6 Hz, 3H). ¹³C NMR (100 MHz, CDCl₃) δ 139.8 (2C), 129.2 (4C), 128.5 (4C), 127.3 (2C), 124.6 (t, J=243.3 Hz), 70.9, 70.2 (t, J=28.3 Hz), 57.2, 54.8, 33.0, 32.8 (t, J=23.8 Hz), 32.1, 29.9 (4C), 29.8, 29.7, 29.6, 29.6, 22.9, 21.5, 14.3, 8.75. ¹⁹F NMR (376 MHz, CDCl₃) 6-111.6 (m, 1F), −113.6 (m, 1F). HRMS (APCI) m/z=518.38041 (Theo. for C₃₂H₄₉F₂NO₂+H: 518.38041). Anal. Calcd. for C₃₂H₄₇F₂NO₂: C, 74.24; H, 9.54; N, 2.71; F, 7.34. Found: C, 74.38; H, 9.60; N, 2.85; F, 7.18.

### (2S,3R,5S)-2-(dibenzylamino)-6,6-difluorooctadecane-3,5-diol

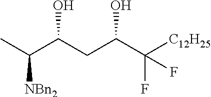

(0.150 g, 0.290 mmol, 40%) R_{f}=0.60 (1:5 EA/H), R_{f}=0.44 (1:10 EA/H). [α]_{D}24=+23.5° (c=1.00 in EtOAc). IR (νₘₐₓ, cm⁻¹) 3475, 2924, 2852, 1454, 1143, 1110, 1075, 1057, 1027, 980, 748, 732, 699. ¹H NMR (400 MHz, CDCl₃) δ 7.25-7.36 (m, 10H), 4.90 (br s, 1H), 4.34 (br s, 1H), 3.98 (m, 1H), 3.83 (d, J=13.2 Hz, 2H), 3.70 (m, 1H), 3.33 (d, J=13.2 Hz, 2H), 2.61 (m, 1H), 1.91 (m, 2H), 1.87 (m, 2H), 1.506 (m, 2H), 1.27 (m, 18H), 1.07 (d, J=6.8 Hz, 3H), 0.897 (t, J=6.8 Hz, 3H). ¹³C NMR (100 MHz, CDCl₃) δ 138.7 (2C), 129.2 (4C), 128.8 (4C), 127.6 (2C), 124.0 (t, J=242.6 Hz), 73.3 (t, J=30.1 Hz), 71.7, 58.9, 53.4, 32.9, 32.7 (t, J=23.8 Hz), 32.1, 29.8 (4C), 29.7, 29.7, 29.6, 29.5, 22.9, 21.7, 14.3, 8.16. ¹⁹F NMR (376 MHz, CDCl₃) 6-110.1 (m, 1F), −115.1 (m, 1F). HRMS (APCI) m/z=518.38066 (Theo. for C₃₂H₄₉F₂NO₂+H: 518.38041). Anal. Calcd. for C₃₂H₄₇F₂NO₂: C, 74.24; H, 9.54; N, 2.71; F, 7.34. Found: C, 73.94; H, 9.61; N, 2.70; F, 7.17.

### (2S,3R,5R)-2-(dibenzylamino)-6,6-difluorooctadecane-3,5-diol

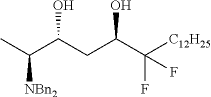

(0.094 g, 0.182 mmol, 25%) R_{f}=0.43 (1:5 EA/H), R_{f}=0.24 (1:10 EA/H). [α]_{D}²⁴=+5.5° (c=1.00 in EtOAc). IR (νₘₐₓ, cm⁻¹) 3399, 2923, 2852, 1454, 1143, 1093, 1058, 1027, 983, 748, 732, 698, 627. ¹H NMR (400 MHz, CDCl₃) δ 7.26-7.35 (m, 10H), 4.70 (br s, 1H), 2.85 (d, J=13.2 Hz, 2H), 3.82 (m, 1H), 3.34 (d, J=13.2 Hz, 2H), 3.17 (app d, J=4.8 Hz, 1H), 2.68 (m, 1H), 1.85 (m, 2H), 1.82 (m, 1H), 1.48 (m, 1H), 1.45 (m, 2H), 1.27 (m, 18H), 1.05 (d, J=6.8 Hz, 3H), 0.890 (t, J=7.0 Hz, 3H). ¹³C NMR (100 MHz, CDCl₃) δ 138.8 (2C), 129.2 (4C), 128.8 (4C), 127.6 (2C), 124.4 (t, J=243.3 Hz), 69.7 (t, J=29.4 Hz), 68.1, 57.9, 53.5, 32.9 (t, J=23.9 Hz), 32.5, 32.1, 29.8 (4C), 29.7, 29.7, 29.6, 29.6, 22.8, 21.6, 14.3, 8.20. ¹⁹F NMR (376 MHz, CDCl₃) 6-111.3 (m, 1F), −114.3 (m, 1F). HRMS (APCI) m/z=518.38074 (Theo. for C₃₂H₄₉F₂NO₂+H: 518.38041). Anal. Calcd. for C₃₂H₄₇F₂NO₂: C, 74.24; H, 9.54; N, 2.71; F, 7.34. Found: C, 74.16; H, 9.68; N, 2.72; F, 7.10.

### (2S,3S,5R)-2-amino-6,6-difluorooctadecane-3,5-diol (ESPD-01406)

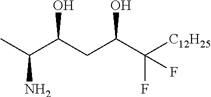

Solution of 2S-(dibenzylamino)-6,6-difluorooctadecane-3,5-diol (1.0 eq) in EtOH was added to a flask with a stir bar. 20% palladium hydroxide on carbon (0.2 eq) was added, and the resulting mixture was stirred vigorously under Ar at room temperature. The reaction mixture was placed under vacuum aspiration for 5 min, and then flushed with Ar. These two steps were repeated twice more. The reaction mixture was placed under vacuum aspiration one last time, and finally was flushed with H₂ via balloon. The reaction was allowed to stir at room temperature overnight under H₂. The next day, LCMS indicated consumption of both starting material and mono-benzyl intermediate. The reaction contents were filtered over celite, which was subsequently washed with methanol. Solvent was evaporated under reduced pressure. Purification was carried out via column chromatography using 86:15:1 DCM:MeOH:NH₄OH to yield white solid (0.199 g, 0.590 mmol, 75%). R_{f}=0.32 (86:15:1 DCM:MeOH:NH₄OH); R_{f}=0.14 (89:10:1 CHCl₃:MeOH:NH₄OH). Mp 117-119° C. [α]_{D}²⁴=−5.5° (c=1.00 in EtOH). IR (νₘₐₓ, cm⁻¹) 3250, 2917, 2850, 1468, 1383, 1216, 1167, 1104, 1064, 1034, 1000, 963, 931, 712, 641. ¹H NMR (400 MHz, CD₃OD) δ 3.88 (m, 1H), 3.79 (m, 1H), 3.03 (m, 1H), 1.91 (m, 2H), 1.84 (m, 1H), 1.67 (m, 1H), 1.51, m (2H), 1.30 (m, 18H), 1.12 (d, J=6.8 Hz, 3H), 0.902 (t, J=6.8 Hz, 3H). ¹³C NMR (100 MHz, CD₃OD) δ 125.5 (t, J=244.1 Hz), 71.4 (t, J=28.2 Hz), 70.1, 51.9, 33.8, 33.5, 33.2, 30.9, 30.9 (2C), 30.8, 30.8, 30.7, 30.6, 23.9, 22.6, 14.6, 12.6. ¹⁹F NMR (376 MHz, CD₃OD) 6-107.0 (m, 1F), −111.0 (m, 1F). HRMS (APCI) m/z=338.28651 (Theo. for C₁₈H₃₇F₂NO₂+H: 338.28651).

### (2S,3S,5S)-2-amino-6,6-difluorooctadecane-3,5-diol (ESPD-01407)

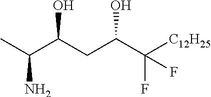

(0.060 g, 0.178 mmol, 75%) R_{f}=0.21 (62:38:2 DCM:MeOH:NH₄OH); R_{f}=0.18 (70:30:2 DCM:MeOH:NH₄OH). Mp 119-122° C. [α]_{D}²⁴=+19.8° (c=1.00 in EtOH). IR (νₘₐₓ, cm⁻¹) 3317, 2917, 2850, 2354, 1089, 1064, 1031, 1008, 976, 962, 683, 645. ¹H NMR (400 MHz, CD₃OD) δ 3.91 (m, 1H), 3.87 (m, 1H), 3.09 (m, 1H), 1.90 (m, 2H), 1.65 (m, 1H), 1.55 (m, 1H), 1.51 (m, 2H), 1.30 (m, 18H), 1.16 (d, J=7.2 Hz, 3H), 0.902 (t, J=6.8 Hz, 3H). ¹³C NMR (100 MHz, CD₃OD) δ 125.8 (t, J=243.3 Hz), 69.9 (t, J=28.7 Hz), 69.0, 53.3, 34.0, 33.9 (t, J=24.5 Hz), 33.2, 30.9, 30.9 (2C), 30.8 (2C), 30.7, 30.6, 23.8, 22.7, 14.6, 13.6. ¹⁹F NMR (376 MHz, CD₃OD) 6-107.3 (m, 1F), −110.9 (m, 1F). HRMS (APCI) m/z=338.28633 (Theo. for C₁₈H₃₇F₂NO₂+H: 338.28651). Anal. Calcd. for C₁₈H₃₇F₂NO₂: C, 64.06; H, 11.05; N, 4.15; F, 11.26. Found: C, 60.72; H, 10.50; N, 3.92; F, 10.28.

### (2S,3R,5S)-2-amino-6,6-difluorooctadecane-3,5-diol (ESPD-01409)

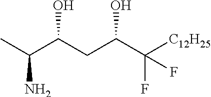

(0.043 g, 0.127 mmol, 44%) R_{f}=0.44 (86:15:1 DCM:MeOH:NH₄OH); R_{f}=0.27 (89:10:1 CHCl₃:MeOH:NH₄OH). Mp 79-82° C. [α]_{D}²⁴=+5.9° (c=1.00 in EtOH). IR (νₘₐₓ, cm⁻¹) 3369, 2922, 2853, 1461, 1381, 1259, 1152, 1087, 961, 858, 806, 721, 666. ¹H NMR (400 MHz, CD₃OD) δ 3.93 (m, 1H), 3.59 (m, 1H), 2.82 (m, 1H), 1.92 (m, 2H), 1.91 (m, 1H), 1.60 (m, 1H), 1.50 (m, 2H), 1.30 (m, 18H), 1.11 (d, J=6.4 Hz, 3H), 0.903 (t, J=6.8 Hz, 3H). ¹³C NMR (100 MHz, CD₃OD) δ 125.7 (t, J=242.9 Hz), 75.3, 71.9 (t, J=29.0 Hz), 52.2, 34.8, 33.7 (t, J=24.6 Hz), 33.2, 31.0, 30.9 (3C), 30.8, 30.7, 30.6, 23.9, 22.7, 19.5, 14.6. ¹⁹F NMR (376 MHz, CD₃OD) 6-107.3 (m, 1F), −111.3 (m, 1F). HRMS (APCI) m/z=338.28622 (Theo. for C₁₈H₃₇F₂NO₂+H: 338.28651). Anal. Calcd. for C₁₈H₃₇F₂NO₂: C, 64.06; H, 11.05; N, 4.15; F, 11.26. Found: C, 64.72; H, 11.00; N, 3.92; F, 10.29.

### (2S,3R,5R)-2-amino-6,6-difluorooctadecane-3,5-diol (ESPD-01408)

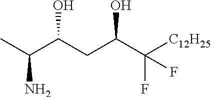

(0.050 g, 0.148 mmol, 82%) R_{f}=0.47 (86:15:1 DCM:MeOH:NH₄OH); R_{f}=0.22 (89:10:1 CHCl₃:MeOH:NH₄OH). Mp 69-72° C. [α]_{D}²⁴=−21.2° (c=1.00 in EtOH). IR (νₘₐₓ, cm⁻¹) 3405, 2916, 2850, 2361, 1469, 1090, 1072, 1053, 1008, 974, 950, 932, 717, 699, 654. ¹H NMR (400 MHz, CD₃OD) δ 3.92 (m, 1H), 3.57 (m, 1H), 3.35 (s, 1H), 2.84 (app p, J=6.2 Hz, 1H), 1.91 (m, 2H), 1.63 (app t, J=6.4 Hz, 1H), 1.51 (m, 2H), 1.30 (m, 18H), 1.13 (d, J=6.0 Hz, 3H), 0.902 (t, J=6.8 Hz, 3H). ¹³C NMR (100 MHz, CD₃OD) δ 125.9 (t, J=243.3 Hz), 72.4, 70.1 (t, J=29.0 Hz), 53.2, 35.0, 33.9 (t, J=24.5 Hz), 33.2, 31.0, 30.9 (2C), 30.8 (2C), 30.7, 30.6, 23.9, 22.8, 18.6, 14.6. ¹⁹F NMR (376 MHz, CD₃OD) 6-108.0 (m, 1F), −111.4 (m, 1F). HRMS (APCI) m/z=338.28616 (Theo. for C₁₈H₃₇F₂NO₂+H: 338.28651). Anal. Calcd. for C₁₈H₃₇F₂NO₂: C, 64.06; H, 11.05; N, 4.15; F, 11.26. Found: C, 62.58; H, 10.70; N, 4.09; F, 10.10.

1. A compound comprising formula I 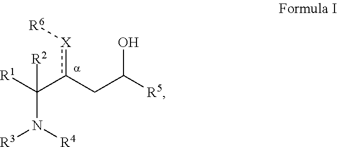
or prodrugs, esters, or salts thereof wherein,
X is O or N;
the dotted line is an optional bond to provide an absent, single, or double bond provided that if X is O and the bond between the X and the alpha carbon is a double bond, then R6 is absent;
R¹ and R² are independently hydrogen or alkyl optionally substituted with one or more, the same or different, R⁷, or R¹ and R² form a 3-7 membered carbocyclic or heterocyclic ring optionally substituted with one or more, the same or different, R⁷;
R³ and R⁴ are independently hydrogen, alkyl, or alkanoyl optionally substituted with one or more R⁷, or R¹ and R³ and the atoms which they are attached form a 4-7 membered heterocyclic ring optionally substituted with one or more, the same or different, R⁷;
R⁵ is a higher alkyl or other lipophilic moiety optionally substituted with one or more, the same or different, R⁷;
R⁶ is hydrogen or alkyl wherein R⁶ is optionally substituted with one or more, the same or different, R7;
R⁷ alkyl, halogen, nitro, cyano, hydroxy, amino, mercapto, formyl, carboxy, carbamoyl, alkoxy, alkanoyl, alkylthio, alkylamino, (alkyl)2amino, alkylsulfmyl, alkylsulfonyl, arylsulfonyl, carbocyclyl, aryl, or heterocyclyl, wherein R⁷ is optionally substituted with one or more, the same or different, R⁸; and R⁸ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfmyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

2. The compound of claim 1, wherein R¹ and R² are alkyl.

3. The compound of claim 1, wherein R⁵ is an unsaturated higher alkyl.

4. The compound of claim 1, wherein R⁵ is a higher alkyl substituted with one or more halogen.

5. The compound of claim 1, wherein R⁵ is alkyl substituted with one or more fluorine.

6. The compound of claim 1, wherein R³ is hydrogen and R⁴ is alkyl.

7. The compound of claim 1 selected from:
2-aminooctadecane-3,5-diol;
(2S,3S,5S)-2-aminooctadecane-3,5-diol;
(2S,3R,5S)-2-aminooctadecane-3,5-diol;
2-(methylamino)octadecane-3,5-diol;
(2S,3R,5S)-2-(methylamino)octadecane-3,5-diol;
2-(dimethylamino)octadecane-3,5-diol;
(2R,3S,5S)-2-(dimethylamino)octadecane-3,5-diol;
1-(pyrrolidin-2-yl)hexadecane-1,3-diol;
(1S,3S)-1-((S)-pyrrolidin-2-yl)hexadecane-1,3-diol; 2-amino-11,11-difluorooctadecane-3,5-diol;
(2S,3 S,5 S)-2-amino-11,11-difluorooctadecane-3,5-diol;
11,11-difluoro-2-(methylamino)octadecane-3,5-diol;
(2S,3S,5S)-11,11-difluoro-2-(methylamino)octadecane-3,5-diol;
N-((2S,3S,5S)-3,5-dihydroxyoctadecan-2-yl)acetamide;
N-((2S,3S,5S)-3,5-dihydroxyoctadecan-2-yl)palmitamide; and
prodrugs, esters, or salts thereof.

8. The compound of claim 1 selected from:
1-(1-aminocyclopropyl)hexadecane-1,3-diol;
(1S,3R)-1-(1-aminocyclopropyl)hexadecane-1,3-diol;
(1S,3S)-1-(1-aminocyclopropyl)hexadecane-1,3-diol;
2-amino-2-methyloctadecane-3,5-diol;
(3S,5S)-2-amino-2-methyloctadecane-3,5-diol;
(3S,5R)-2-amino-2-methyloctadecane-3,5-diol;
(3S,5S)-2-methyl-2-(methylamino)octadecane-3,5-diol;
2-amino-5-hydroxy-2-methyloctadecan-3-one; and
(Z)-2-amino-5-hydroxy-2-methyloctadecan-3-one oxime;
prodrugs, esters, or salts thereof.

9. The compound of claim 1 selected from:
(2S,3R,5R)-2-amino-6,6-difluorooctadecane-3,5-diol;
(2S,3S,5R)-2-amino-6,6-difluorooctadecane-3,5-diol;
(2S,3S,5S)-2-amino-6,6-difluorooctadecane-3,5-diol;
(2S,3R,5S)-2-amino-6,6-difluorooctadecane-3,5-diol;
(2S,3S,5S)-2-amino-18,18,18-trifluorooctadecane-3,5-diol; and
prodrugs, esters, or salts thereof.

10. A pharmaceutical composition comprising a compound of claim 1 or salt thereof and a pharmaceutically acceptable excipient.

11. A pharmaceutical composition of claim 10, further comprising a second therapeutic agent.

12. The pharmaceutical composition of claim 11, wherein the second therapeutic agent is an anti-malaria agent, anti-viral agent, antibiotic, or anti-cancer agent.

13. A method of treating or preventing an infection comprising administering an effective amount of a compound in claim 1 to a subject in need thereof.

14. The method of claim 13, wherein the subject is diagnosed with or at risk of a malaria infection.

15. The method of claim 13, wherein the subject is diagnosed with or at risk of an infection from a virus, bacteria, fungi, protozoa, or parasite.

16. The method of claim 13, wherein the compound is administered in combination with a second therapeutic agent.

17. The method of claim 16, wherein the second therapeutic agent is an anti-malaria agent, anti-viral agent, or antibiotic.

18. A method of treating or preventing cancer comprising administering an effective amount of a compound of any of claim 1 to a subject in need thereof.

19. The method of claim 18, wherein the cancer is selected from bladder cancer, lung cancer, breast cancer, melanoma, colon and rectal cancer, non-hodgkin lymphoma, endometrial cancer, pancreatic cancer, kidney cancer, prostate cancer, leukemia, thyroid cancer, and brain cancer.

20. The method of claim 18, wherein the compound is administered in combination with a second anti-cancer agent.
